SEQUENCE LISTING (20 sequences)

SEQ ID NO: 1 - DNA, 19 bp, Artificial Sequence
  primer sequences of Col1a1(forward)
ctggcggttcaggtccaat

SEQ ID NO: 2 - DNA, 19 bp, Artificial Sequence
  primer sequences of Col1a1(reverse)
ttccaggcaatccacgagc

SEQ ID NO: 3 - DNA, 22 bp, Artificial Sequence
  primer sequences of Col3a1(forward)
acgtagatgaattgggatgcag

SEQ ID NO: 4 - DNA, 21 bp, Artificial Sequence
  primer sequences of Col3a1(reverse)
gggttggggcagtctagtggc

SEQ ID NO: 5 - DNA, 22 bp, Artificial Sequence
  primer sequences of Timp1(forward)
gggttccccagaaatcaacgag

SEQ ID NO: 6 - DNA, 25 bp, Artificial Sequence
  primer sequences of Timp1(reverse)
acagaggctttccatgactggggtg

SEQ ID NO: 7 - DNA, 22 bp, Artificial Sequence
  primer sequences of Tgf-beta-1(forward)
gtgaaacggaagcgcatcgaag

SEQ ID NO: 8 - DNA, 22 bp, Artificial Sequence
  primer sequences of Tgf-beta-1(reverse)
acgtagatgaattgggatgcag

SEQ ID NO: 9 - DNA, 21 bp, Artificial Sequence
  primer sequences of Hgf(forward)
ctgacccaaacatccgagttg

SEQ ID NO: 10 - DNA, 21 bp, Artificial Sequence
  primer sequences of Hgf(reverse)
ttcccattgccacgataacaa

SEQ ID NO: 11 - DNA, 20 bp, Artificial Sequence
  primer sequences of Igf(forward)
tgcttccggagctgtgatct

SEQ ID NO: 12 - DNA, 19 bp, Artificial Sequence
  primer sequences of Igf(reverse)
cgggctgcttttgtaggct

SEQ ID NO: 13 - DNA, 19 bp, Artificial Sequence
  primer sequences of Ang1(forward)
gggacagcaggcaaacaga

SEQ ID NO: 14 - DNA, 22 bp, Artificial Sequence
  primer sequences of Ang1(reverse)
tgtcgttatcagcatccttcgt

SEQ ID NO: 15 - DNA, 20 bp, Artificial Sequence
  primer sequences of Vegfa(forward)
gcaggctgctgtaacgatga

SEQ ID NO: 16 - DNA, 22 bp, Artificial Sequence
  primer sequences of Vegfa(reverse)
gcatgatctgcatggtgatgtt

SEQ ID NO: 17 - DNA, 19 bp, Artificial Sequence
  primer sequences of Hif1-alpha(forward)
acaagtcaccacaggacag

SEQ ID NO: 18 - DNA, 18 bp, Artificial Sequence
  primer sequences of Hif1-alpha(reverse)
agggagaaaatcaagtcg

SEQ ID NO: 19 - DNA, 24 bp, Artificial Sequence
  primer sequences of Hif2-alpha(forward)
aatgacagctgacaaggagaaaaa

SEQ ID NO: 20 - DNA, 24 bp, Artificial Sequence
  primer sequences of Hif2-alpha(reverse)
gagtgaagtcaaagatgctgtgtc

(19) United States
(12) Patent Application Publication
(10) Pub. No.: US 2025/0000909 A1
(43) Pub. Date: Jan. 2, 2025

(54) PHARMACEUTICAL COMPOSITION FOR PREVENTING OR TREATING ASHERMAN'S SYNDROME COMPRISING ISOLATED MITOCHONDRIA AS ACTIVE INGREDIENT

(71) Applicant: Cha University Industry-Academic Cooperation Foundation, Pocheon-si, Gyeonggi-do (KR)

(72) Inventors: Yong-Soo CHOI, Pocheon-si, Gyeonggi-do (KR); Haengseok SONG, Pocheon-si, Gyeonggi-do (KR); Mi Jin KIM, Pocheon-si, Gyeonggi-do (KR); Mira PARK, Pocheon-si, Gyeonggi-do (KR)

(21) Appl. No.: 18/291,073

(22) PCT Filed: Mar. 31, 2022
(86) PCT No.: PCT/KR2022/004637

§ 371 Date: Jan. 22, 2024

(30) Foreign Application Priority Data
Jul. 23, 2021 (KR) 10-2021-0097096

(51) Int. Cl. A61K 35/28; A61P 15/00

(52) U.S. Cl. CPC A61K 35/28; A61P 15/00

(57) ABSTRACT

Provided is a pharmaceutical composition for preventing or treating Asherman's syndrome or complications thereof, comprising mitochondria as an active ingredient. The pharmaceutical composition alleviates or treats endometrial adhesion, and may alleviate or treat endometrial fibrosis. Therefore, the pharmaceutical composition may be effectively used in the treatment and prevention of diseases such as endometrial adhesion disease, particularly Asherman's syndrome, and thus is highly industrially applicable.

### TECHNICAL FIELD

The present invention relates to a pharmaceutical composition for preventing or treating Asherman's syndrome or complications thereof, comprising isolated mitochondria as an active ingredient.

### BACKGROUND ART

Asherman's syndrome, also known as intrauterine adhesions (IUAs), is caused by deprivation and defects of the endometrium basal layer and partial or extensive adhesions in the uterine cavity. Symptoms of Asherman's syndrome include sterility, habitual miscarriage or premature birth, amenorrhea, hypomenorrhea, or dysmenorrhea. Known treatment methods for Asherman's syndrome include adhesiolysis by transvaginal or laparotomy, insertion of an intrauterine contraceptive device (IUD), and administration of hormones. However, many intrauterine re-adhesions occur after surgery, so multiple surgeries are required, and in severe cases, there were problems such as the possibility of sterility due to repeated surgery.

In addition, known treatment methods for Asherman's syndrome include conservative therapy using analgesics and anti-inflammatory agents to relieve pain, or hormone treatment therapy that controls the menstrual cycle using danazol, progesterone, or gonadotropin-releasing hormone (GnRH). However, these have limitations in improving symptoms rather than fundamental treatments, and long-term use of hormones causes various side effects (weight gain, moisture accumulation, fatigue, acne, oily skin, hypertrichosis, atrophic vaginitis, facial flushing, muscle cramps, unstable emotional state, and hepatocellular toxicity), and the recurrence rate is very high (Chinese Patent Application Publication No. 107073040).

These intrauterine adhesions are a disease that causes discomfort in daily life due to extreme pain and, in severe cases, can lead to sterility. Nevertheless, there is still no fundamental prevention or treatment method, and related research is also lacking.

Meanwhile, mitochondria are an organelle essential for the survival of eukaryotic cells that are involved in the synthesis and regulation of adenosine triphosphate (ATP) as an energy source. Mitochondria are an important organelle involved in various metabolic pathways in vivo, such as cell signaling, cell differentiation, and cell death, as well as the control of the cell cycle and cell growth. There has been no research on whether a pharmaceutical composition comprising these mitochondria as an active ingredient may be related to the treatment of intrauterine adhesion.

### DETAILED DESCRIPTION OF INVENTION

### Technical Problem

Accordingly, the present inventors have developed a composition for the treatment of Asherman's syndrome or complications thereof comprising isolated mitochondria as an active ingredient for the treatment of Asherman's syndrome or complications thereof, thereby developing a method for fundamentally treating Asherman's syndrome or complications thereof to solve the above problems.

### Solution to Problem

In one aspect of the present invention, there is provided a pharmaceutical composition for preventing or treating Asherman's syndrome or complications thereof, comprising isolated mitochondria as an active ingredient.

In another aspect of the present invention, there is provided a treatment method for preventing or treating Asherman's syndrome or complications thereof, comprising administering the pharmaceutical composition according to any one claim to a subject.

In another aspect of the present invention, there is provided the use of isolated mitochondria for the prevention or treatment of Asherman's syndrome or complications thereof.

### Effects of Invention

A pharmaceutical composition comprising mitochondria as an active ingredient may induce the proliferation of vascular cells in the endometrium and suppress inflammation. Therefore, uterine disease induced by uterine fibrosis may be effectively treated or prevented. Therefore, the pharmaceutical composition may be usefully used to effectively treat Asherman's syndrome or sterility, infertility, and premature birth caused by Asherman's syndrome.

### BRIEF DESCRIPTION OF DRAWINGS

FIG. 1 is a graph showing the amount of protein in stem cell-derived mitochondria.

FIG. 2 is a graph showing the size distribution of stem cell-derived mitochondria.

FIG. 3 is an image of stem cell-derived mitochondria observed under a fluorescence microscope.

FIG. 4 is a graph confirming stem cell-derived mitochondria through flow cytometry.

FIG. 5 is an image showing the purity of mitochondria using mitochondria markers.

FIG. 6 is a graph confirming the ATP synthesis ability of stem cell-derived mitochondria.

FIG. 7 is a graph showing ROS production in stem cell-derived mitochondria.

FIG. 8 is a graph showing the membrane potential of stem cell-derived mitochondria.

FIG. 9 is a graph showing the ATP synthesis ability of stem cell-derived mitochondria.

FIG. 10 is a schematic diagram of an experiment to confirm changes in fibrosis status and histological appearance in an Asherman's syndrome mouse model (AS) by administration of stem cell-derived mitochondria (MT).

FIG. 11 is an image showing the degree of abnormality in fibrosis status and histological appearance in an Asherman's syndrome mouse model (AS) by administration of stem cell-derived mitochondria (MT).

FIG. 12 is a graph showing the mRNA expression of fibrosis factors (Col1a1, Col3a1, Timp1, and Tgfβ1) by administration of stem cell-derived mitochondria (MT).

FIG. 13 is a graph showing the protein expression of fibrosis factors (Col1a1, Col3a1, Timp1, and Tgfβ1) by administration of stem cell-derived mitochondria (MT).

FIG. 14 is a schematic diagram of an experiment to confirm pregnancy-related indicators in an Asherman's syndrome mouse model (AS) by administration of stem cell-derived mitochondria (MT).

FIG. 15 is a graph showing the number of implanted embryos in the second trimester of pregnancy in an Asherman's syndrome mouse model (AS) by administration of stem cell-derived mitochondria (MT).

FIG. 16 is a graph showing the weight of implanted embryos in the second trimester of pregnancy in an Asherman's syndrome mouse model (AS) by administration of stem cell-derived mitochondria (MT).

FIG. 17 is a graph showing the time to conceive in an Asherman's syndrome mouse model (AS) by administration of stem cell-derived mitochondria (MT).

FIG. 18 is a graph showing the delivery rate at the end of pregnancy in an Asherman's syndrome mouse model (AS) by administration of stem cell-derived mitochondria (MT).

FIG. 19 is a graph showing the litter size at the end of pregnancy in an Asherman's syndrome mouse model (AS) by administration of stem cell-derived mitochondria (MT).

FIG. 20 is a graph showing the number of implanted uteri and implanted embryos in the early stage of pregnancy in an Asherman's syndrome mouse model (AS) by administration of stem cell-derived mitochondria (MT).

FIG. 21 is a graph showing the mRNA expression of vascular endothelial cell markers Hgf, Igf1, Ang1, Vegfa, Hif1α, and Hif2α by administration of stem cell-derived mitochondria (MT).

FIG. 22 is an image showing the protein expression of vascular endothelial cell markers Hgf, Igf1, Ang1, Vegfa, Hif1α, and Hif2α by administration of stem cell-derived mitochondria (MT).

FIG. 23 is an immunofluorescence staining image showing the cell proliferation effect by administration of stem cell-derived mitochondria (MT).

FIG. 24 is a graph quantitatively showing the ratio of KI-67⁺ expressing proliferating cells among CD31⁺ expressing vascular cells in the endometrium administered with stem cell-derived mitochondria (MT).

FIG. 25 is a graph showing the mRNA expression of fibrosis factors (Col1a1, Col3a1, Timp1, and Tgfβ1) when dead MT and live MT were injected under the same conditions.

FIG. 26 is a graph showing the mRNA expression of fibrosis factors (Col1a1, Col3a1, Timp1, and Tgfβ1) upon administration of stem cell-derived mitochondria (MT) by injection method and injection dose.

FIG. 27 is a schematic diagram of an experiment to confirm the effect of intravenous injection of stem cell-derived mitochondria (MT).

FIG. 28 is an image showing the effect of improving uterine fibrosis upon intravenous injection of stem cell-derived mitochondria (MT).

FIG. 29 is a graph showing real-time RT-PCR results for fibrosis-related factors after delivery of stem cell-derived mitochondria (MT).

FIG. 30 is an image showing changes in CD45 expression of immune cells by administration of stem cell-derived mitochondria (MT).

FIG. 31 is an image showing the ratio of total macrophages expressing F4/80, the ratio of M1 macrophages expressing CD80, and the ratio of M2 macrophages expressing CD206 by administration of stem cell-derived mitochondria (MT).

FIG. 32 is a schematic diagram of an experiment to confirm the effect of macrophage depletion.

FIG. 33 is a graph showing the number of macrophages after depletion of macrophages expressing F4/80.

FIG. 34 is an image showing through fluorescence staining that there is no effect of endometrial regeneration by MT in a macrophage-depleted environment.

FIG. 35 is a graph showing through changes in mRNA expression that there is no effect of endometrial regeneration by MT in a macrophage-depleted environment.

FIG. 36 is a schematic diagram of an experiment to confirm M2 polarization of macrophages by treatment with stem cell-derived mitochondria (MT).

FIG. 37 is an image showing M2 polarization of macrophages by treatment with stem cell-derived mitochondria (MT).

FIG. 38 is a graph showing changes in expression of M1 markers iNOS and Socs3, and M2 markers Arg1 and Mrc1 of macrophages by treatment with stem cell-derived mitochondria (MT).

FIG. 39 is an immunocytochemical image showing M2 polarization of macrophages by treatment with stem cell-derived mitochondria (MT).

FIG. 40 is a flow cytometry graph showing M2 polarization of macrophages by treatment with stem cell-derived mitochondria (MT).

FIG. 41 is a schematic diagram of an experiment to confirm whether M2 macrophages polarized by administration of stem cell-derived mitochondria (MT) promote the formation and migration of human umbilical vein endothelial cells (HUVEC).

FIG. 42 is an image showing the migration speed in each experimental group when polarized M2 macrophages and HUVEC were co-cultured.

FIG. 43 is an image showing the degree of angiogenesis in each experimental group when polarized M2 macrophages and HUVEC were co-cultured.

FIG. 44 is a graph showing the degree of angiogenesis in each experimental group when polarized M2 macrophages and HUVEC were co-cultured.

FIG. 45 is a schematic diagram of the functional improvement of damaged endometrium by stem cell-derived mitochondria.

FIG. 46 is a graph showing the protein content of hepatocyte-derived mitochondria.

FIG. 47 is a graph showing the protein content of peripheral blood mononuclear cell-derived mitochondria.

FIG. 48 is a graph showing the ATP synthesis ability of hepatocyte-derived mitochondria.

FIG. 49 is a graph showing the ATP synthesis ability of peripheral blood mononuclear cell-derived mitochondria.

### MODE FOR CARRYING OUT THE INVENTION

### A Therapeutic Agent for Asherman's Syndrome or Complications Thereof Comprising Isolated Mitochondria as an Active Ingredient

In one aspect of the present invention, there is provided a pharmaceutical composition for preventing or treating Asherman's syndrome or complications thereof, comprising isolated mitochondria as an active ingredient.

As used herein, the term “mitochondria” is a double membrane-bound organelle found in most eukaryotes that produces the majority of intracellular adenosine triphosphate (ATP).

As used herein, the term “isolated mitochondria” refers to mitochondria obtained from autologous, allogeneic, or xenogeneic sources.

As used herein, the term “autologous mitochondria” refers to mitochondria obtained from plasma, tissue, bone marrow, or cells of the same subject. In addition, the term “allogeneic mitochondria” refers to mitochondria obtained from plasma, tissue, bone marrow, or cells of a subject belonging to the same species as the subject and having a different genotype with respect to alleles. In addition, the term “xenogeneic mitochondria” refers to mitochondria obtained from plasma, tissue, bone marrow, or cells of a subject belonging to a different species than the subject.

In this case, the subject may be a mammal, and preferably may be a human.

The mitochondria may be isolated from cells, bone marrow, or plasma of a subject. The mitochondria may be obtained from autologous or allogeneic cells cultured in vitro. In this case, the cells, bone marrow, or plasma may have normal biological activity.

As used herein, the term “cell” refers to a structural or functional unit constituting a living organism, consisting of cytoplasm surrounded by a cell membrane, and comprising biomolecules such as proteins and nucleic acids. The cell may be a cell comprising mitochondria inside the cell membrane.

In addition, the mitochondria may be used by concentrating tissue, plasma, bone marrow, or cells, disrupting them, and then isolating them or may be disrupted; or isolated from tissue, plasma, bone marrow, or cell samples that have been frozen-stored and then thawed.

In one embodiment, the cells may be any one selected from the group consisting of stem cells, somatic cells, germ cells, and platelets.

As used herein, the term “stem cell” refers to an undifferentiated cell that has the ability to differentiate into various types of tissue cells. The stem cells may be any one selected from the group consisting of mesenchymal stem cells, adult stem cells, induced pluripotent stem cells, embryonic stem cells, bone marrowstem cells, neural stem cells, limbal stem cells, and tissue-derived stem cells.

In this case, the mesenchymal stem cells may be derived from any one selected from the group consisting of umbilical cord, umbilical cord blood, bone marrow, fat, muscle, nerve, skin, amniotic membrane, and placenta. Preferably, it may be derived from human umbilical cord.

As used herein, the term “somatic cell” refers to cells that make up an organism, excluding germ cells. The somatic cell may be one selected from the group consisting of muscle cells, hepatocytes, fibroblasts, epithelial cells, nerve cells, adipocytes, osteocytes, periosteal cells, white blood cells, lymphocytes, and mucosal cells. Preferably, it may be obtained from muscle cells or hepatocytes with excellent mitochondrial activity. In addition, it may be obtained from autologous or allogeneic blood PBMC (peripheral blood mononuclear cell) cells.

As used herein, the term “germ cell” refers to a cell that forms a zygote during reproduction in an organism that reproduces sexually. The mitochondria may be obtained from autologous or allogeneic germ cells. The germ cells may be sperm or eggs.

As used herein, the term “platelet” refers to a solid component that plays an important role in blood coagulation by binding fibrin in the blood to form a blood clot. The mitochondria may be obtained from autologous or allogeneic platelets.

As used herein, the term “bone marrow” refers to the semi-solid tissue found in the spongy portion of the bone. The bone marrow in humans produces about 500 billion blood cells per day. In particular, the bone marrow comprises mitochondria with normal activity.

As used herein, the term “plasma” refers to the liquid component of blood, excluding blood cells, and the intravascular portion of extracellular fluid. The plasma comprises up to 95% of water and 6 to 8% of dissolved proteins or electrolytes. In particular, the plasma comprises mitochondria with normal activity.

The plasma may be obtained by separating it from the blood. Specifically, blood comprising an anticoagulant may be spun in a centrifuge to isolate the supernatant from the blood. In addition, plasma may be extracted from the blood through filtration or coagulation. In addition, the plasma may be classified according to the blood from which it is derived. In one embodiment, the plasma may be plasma isolated from umbilical cord blood or peripheral blood. Preferably, it may be separated from umbilical cord blood.

In one embodiment, the plasma or bone marrow may be obtained and stored from a subject. Specifically, the plasma or bone marrow may be frozen.

In addition, the isolated mitochondria may have normal biological activity. Specifically, the mitochondria with normal biological activity may have one or more properties selected from the group consisting of (i) having a membrane potential, (ii) generating ATP within the mitochondria, and (iii) removing ROS or reducing the activity of ROS within the mitochondria.

In one embodiment, when the composition of the present invention comprising isolated mitochondria as an active ingredient was administered directly into the uterus or intravenously, intrauterine regeneration was promoted and fibrosis indicators were reduced. Therefore, the composition of the present invention comprising isolated mitochondria as an active ingredient may alleviate or treat intrauterine fibrosis or intrauterine adhesion, and has a preventive or therapeutic effect on complications of Asherman's syndrome.

As used herein, the term “Asherman's syndrome,” also known as intrauterine adhesions (IUAs), refers to the formation of adhesions in the uterus as the endometrial basal layer falls and normal regeneration becomes difficult.

The Asherman's syndrome mainly occurs in patients with a history of surgery such as endometrial curettage, cervical cone biopsy, and electrocautery; patients with a history of pelvic inflammatory disease; and patients with infections caused by intrauterine contraceptive devices, etc., and is characterized by fibrosis of the endometrium, damage to uterine cervix, destruction of the endometrium, and adhesions in the uterine cavity. In addition, Asherman's syndrome in the uterus is generally accompanied by complications, and decreased menstruation, amenorrhea, uterine pain, sterility, infertility, and the like may occur.

As used herein, the term “Asherman's syndrome complication” refers to a disease that may accompany Asherman's syndrome or refers collectively to a disease that may increase the risk of Asherman's syndrome. Specifically, it includes diseases or symptoms that accompany intrauterine adhesion or that may increase the risk of intrauterine adhesion.

In one embodiment, the Asherman's syndrome complication may be one or more selected from the group consisting of intrauterine adhesion, leiomyoma of uterus, endometriosis, ectopic pregnancy, miscarriage, ovarian cystic tumor, menstrual disorder, infertility, sterility, pelvic adhesion, pelvic pain, and pelvic inflammatory disorder, but is not limited thereto.

As used herein, the term “intrauterine adhesion,” also referred to as intrauterine synechiae, refers to a disease in which the endometrium is damaged or the inner walls of the uterus stick together and become hard.

As used herein, the term “leiomyoma of uterus” refers to a benign tumor that occurs in the muscle layer that makes up the uterus. Leiomyoma of uterus is classified as uterine body myoma, cervical myoma, or uterine vaginal myoma based on the location of the myomas. Leiomyoma of uterus may affect sterility or repeated miscarriages.

As used herein, the term “endometriosis” refers to a disease in which endometrial tissue exists outside the uterus, causing the disease. The endometriosis is a disease in which ectopic endometrial cells located outside the uterus may cause bleeding or inflammatory reactions during the menstrual cycle, ultimately leading to fibrosis or adhesion, etc.

As used herein, the term “ectopic pregnancy” refers to implantation of a fertilized egg in a site other than the uterine trunk. More than 95% of ectopic pregnancies are tubal pregnancies that implant in the ampulla, and occur when the fallopian tubes are narrowed due to some factor or the tubal mucosa has an increased capacity for a fertilized egg.

As used herein, the term “menstrual disorder,” also referred to as dysmenorrhea, refers to a disease accompanied by abnormal uterine bleeding, amenorrhea, menstrual pain, early menopause (primary ovarian failure), or premenstrual syndrome during the reproductive period.

As used herein, the term “ovarian cystic tumor” refers to a cyst in the ovary. The cysts are filled with fluid components and are classified into functional cysts and benign ovarian tumors. The ovarian cystic tumor may cause intrauterine adhesion, sterility, or infertility.

As used herein, the term “infertility” or sterility” refers to the inability to become pregnant for more than one year even when engaging in normal sexual activity without contraception, or the inability of a woman over 35 years of age to become pregnant within 6 months when engaging in normal sexual activity without contraception.

As used herein, the term “pelvic adhesion,” also referred to as pelvic organ adhesion, refers to a state in which different tissues or organs are connected and attached by fibrous tissue within the pelvis. The organ may be the uterus, ovaries, fallopian tubes, or peritoneum.

As used herein, the term “pelvic inflammatory disorder (PID),” also referred to as pelvic inflammation, refers to an infection occurring inside the uterus, fallopian tubes, ovaries, or pelvis, which is the upper part of the female reproductive organ. In addition, the term “pelvic pain (chronic pelvic pain)” refers to pain occurring due to inflammation in the pelvis, etc.

As used herein, the term “treatment” may be used to include both therapeutic treatment and preventive treatment. In this case, prevention may be used to mean alleviating or reducing the pathological condition or disease of a subject.

As used herein, the term “active ingredient” refers to an ingredient that exhibits an activity alone or in combination with an auxiliary agent (carrier) that does not has an activity on its own.

The isolated mitochondria as an active ingredient may prevent excessive fibrosis of the uterus or reduce fibrosis of the uterus. Specifically, the mitochondria may reduce the expression of fibrosis factors in the uterus.

As used herein, the term “fibrosis” refers to a phenomenon in which extracellular matrix components such as collagen are excessively accumulated by fibroblasts during repetitive injury, chronic inflammation, or the recovery process thereof. Fibrosis may be caused by macrophages releasing fibrosis factors.

As used herein, the term “fibrosis factor” refers collectively to proteins that stimulate fibroblasts. The fibrosis factor may be one or more selected from the group consisting of COL1A1, COL3A1, TIMP1, and TGFβ1.

The COL1A1 refers to type I collagen present in most connective tissues. Type 1 collagen may be encoded by the COL1A1 gene in humans.

The COL3A1 refers to type III collagen synthesized as pre-procollagen by cells. The type III collagen may be encoded by the COL3A1 gene in humans.

The TIMP1 is a glycoprotein that may promote cell proliferation in a wide range of cell types and has an anti-apoptotic function, and is also referred to as TIMP metallopeptidase inhibitor 1. The TIMP1 may be encoded by the TIMP1 gene in humans.

The TGFβ1 is a member of the transforming growth factor beta superfamily of cytokines. The TGFβ1 performs many cellular functions, including the control of cell growth, cell proliferation, cell differentiation, and cell death. The TGFβ1 may be encoded by the TGFβ1 gene in humans.

In one embodiment, the isolated mitochondria may reduce the expression of one or more proteins selected from the group consisting of COL1A1, COL3A1, TIMP1, and TGFβ1 or genes encoding the same in the uterus.

In addition, the mitochondria may increase vascular endothelial cells. In addition, the mitochondria may increase the ratio of vascular cells proliferating in blood vessels within the uterus. Specifically, it may increase the ratio of cells expressing KI-67 among vascular cells expressing CD31 in blood vessels within the uterus.

In one embodiment, the expression of vascular endothelial cell markers may be increased in the uterus. The vascular endothelial cell marker may be one or more selected from the group consisting of HGF, IGF1, ANG1, VEGF-A, HIF1α, and HIF2α.

The HGF is a hepatocyte growth factor, which refers to a cytokine that increases mitosis, cell motility, and matrix invasion, thereby inducing angiogenesis, tumor formation, and tissue regeneration. The HGF may be encoded by the HGF gene in humans.

The IGF1 is insulin-like growth factor 1, also referred to as somatomedin C, and refers to a protein with high sequence similarity to insulin. The IGF1 may be encoded by the IGF1 gene in humans.

The ANG1 is angiopoietin 1, which refers to a protein that plays an important role in blood vessel development and angiogenesis. The ANG1 may be encoded by the ANGPT1 gene in humans.

The VEGF-A is vascular endothelial growth factor A. The VEGF-A acts specifically on endothelial cells to mediate increased vascular permeability, induce angiogenesis, angiogenesis, and endothelial cell growth, promote cell migration, and inhibit apoptosis. The VEGF-A may be encoded by the VEGFA gene in humans.

The HIF1α is hypoxia-inducible factor 1-alpha, which refers to a protein that induces the transcription of genes encoding VEGF and erythropoietin, which have functions such as angiogenesis and erythropoiesis. The HIF1α promotes and increases oxygen delivery. The HIF1α may be encoded by the HIF1A gene in humans.

The HIF2α is hypoxia-inducible factor 2-alpha, which refers to a protein that improves oxygen transport, and is also referred to as EPAS1 (endothelial PAS domain-containing protein 1). The HIF2α may be encoded by the EPAS1 gene in humans.

In one embodiment, the isolated mitochondria may increase the expression of one or more proteins selected from the group consisting of HGF, IG1F, ANG1, VEGF-A, HIF1α, and HIF2α or genes encoding the same.

The mitochondria may reduce inflammation in the uterus. Specifically, it may reduce the gene expression of inflammatory factors iNOS and SOCS3, or a combination thereof, or may increase the gene expression of anti-inflammatory factors ARG1 and MRC1, or a combination thereof.

The iNOS refers to the inducible isomer of nitric oxide synthases involved in the immune response. The iNOS is an inflammatory factor that produces NO by pro-inflammatory cytokines (e.g., interleukin-1, tumor necrosis factor alpha, and interferon gamma).

The SOCS3 is an inflammatory factor induced by various cytokines including IL-6, IL-10, and interferon (IFN)-gamma in humans.

The ARG1 is a gene encoding arginase protein, and arginase catalyzes the hydrolysis of arginine into ornithine and urea.

The MRC1 is macrophage mannose receptor 1, and is also referred to as CD206. The CD206 is present on the surface of macrophages, and the level of expression may vary depending on the polarization of the macrophages.

The mitochondria may promote the polarization of macrophages in the uterus.

As used herein, the term “macrophage” refers to an immune cell that defends the host from infection through phagocytosis. Macrophages are classified based on their basic function and activation, and classified into activated macrophages (M1 macrophages), wound healing macrophages (M2 macrophages), and regulatory macrophages.

The M1 macrophages are activated by LPS and IFN-gamma and secrete high levels of IL-12 and low levels of IL-10 compared to M2 macrophages. The M1 macrophages promote inflammation and have bactericidal and phagocytic functions. In one embodiment, the M1 macrophages may have high levels of CD80 expression and low levels of CD206 expression compared to M2 macrophages.

The M2 macrophages secrete high levels of IL-10 and low levels of IL-12 compared to M1 macrophages. The M2 macrophages produce anti-inflammatory cytokines to heal wounds and repair tissues. In one embodiment, the M2 macrophages may have low levels of CD80 expression and high levels of CD206 expression compared to M1 macrophages.

In addition, the macrophages may be polarized from M1 macrophages to M2 macrophages by IL-4 cytokine.

In one embodiment, the mitochondria may polarize macrophages in the uterus from M1 macrophages to M2 macrophages. In addition, it may reduce CD80 expression and may increase CD206 expression of macrophages in the uterus.

In addition, the mitochondria may induce uterine regeneration. Specifically, the mitochondria may induce regeneration of the damaged uterine lining by suppressing excessive fibrosis in the uterus, by promoting the formation and migration of blood vessels, by suppressing inflammation, and by promoting the polarization of macrophages.

In addition, the mitochondria may promote the formation of the umbilical cord. Specifically, it may promote the migration of blood vessels or the formation of blood vessels in the umbilical cord of an implanted fetus.

When isolating the mitochondria from specific cells, for example, they may be isolated through various known methods, such as using a specific buffer solution or using a potential difference and magnetic field. In addition, the isolation of mitochondria may include centrifuging and filtering plasma to remove all cellular components, and centrifuging the filtered plasma.

The isolation of mitochondria may be obtained by disrupting and centrifuging cells in terms of maintaining mitochondrial activity. In this case, centrifugation may be performed in the first to third stages.

In one embodiment, it may be performed by culturing cells and performing a first centrifugation of a pharmaceutical composition comprising these cells to produce a pellet, resuspending the pellet in a buffer solution and homogenizing it, performing a second centrifugation of the homogenized solution to prepare a supernatant, and performing a third centrifugation of the supernatant to purify mitochondria. In this case, it is preferable in terms of maintaining cell activity that the time for performing the second centrifugation is adjusted to be shorter than the time for performing the first centrifugation and the third centrifugation, and the speed may be increased from the first centrifugation to the third centrifugation.

When isolating the mitochondria from plasma, for example, they may be isolated through various known methods, such as using a specific buffer solution or using sonication, concentration gradient, and magnetic field.

The isolation of mitochondria includes removing cells or cell organelles from plasma; and purifying mitochondria. In addition, the isolation of mitochondria may include physically isolating the endoplasmic reticulum, mitochondria-related membrane debris, and mitochondria.

In one embodiment, the isolation may be by centrifugation. Specifically, the isolation may be performed by performing a first centrifugation of the plasma at low speed to remove cells in the plasma; filtering the plasma to remove cell debris; and performing a second centrifugation of the plasma supernatant.

In one embodiment, the isolation may be by discontinuous concentration gradient and centrifugation. The discontinuous concentration gradient may use a sucrose or Percoll concentration gradient. Specifically, the isolation may be performed by lysing cells using sonication; performing a first centrifugation of the plasma at low speed to remove cells in the plasma; performing a second centrifugation of the plasma to remove endoplasmic reticulum; loading the supernatant of plasma into a discontinuous concentration gradient; and performing a third centrifugation of the isolated product;

The first centrifugation to the third centrifugation may be performed at a temperature of 0 to 10° C., preferably 3 to 5° C. In addition, the time for which the centrifugation is performed may be 1 to 50 minutes, and may be appropriately adjusted depending on the number of centrifugations and the content of the sample. In addition, the first centrifugation may be performed at a speed of 100 to 1,000×g, or 200 to 700×g, or 300 to 450×g. In addition, the second centrifugation or the third centrifugation may be performed at a speed of 1 to 2,000×g, 25 to 1,800×g, or 500 to 1,600×g, 100 to 20,000×g, 500 to 18,000×g, or 800 to 15,000×g.

The mitochondria may be quantified by quantifying the membrane proteins of the isolated mitochondria. Specifically, the isolated mitochondria may be quantified using BCA (bicinchoninic acid assay) analysis method. In this case, the mitochondria in the pharmaceutical composition may be included at a concentration of 0.1 μg/mL to 1,000 μg/mL, 1 μg/mL to 750 μg/mL, 25 μg/mL to 500 μg/mL, 25 μg/mL to 150 μg/mL, or 25 μg/mL to 100 μg/mL. In one embodiment of the present invention, a concentration of 25 μg/mL or 50 μg/mL was used.

In addition, the mitochondria may have an intact form, a disrupted form, or a combination thereof. In one embodiment, even when the mitochondria are in a disrupted form, they may exhibit pharmacological effects if they have mitochondrial activity.

In addition, the number of the isolated mitochondria may be measured using a particle counter (Multisizer 4e, Beckman Coulter).

In one embodiment, the mitochondria in the pharmaceutical composition may be included in an amount of 1×10⁵ mitochondria/mL to 9×10⁹ mitochondria/mL. Specifically, the mitochondria in the pharmaceutical composition may be included in an amount of 1×10⁵/mL to 5×10⁹, 2×10⁵/mL to 2×10⁹/mL, 5×10⁵/mL to 1×10⁹/mL, 1×10⁶/mL to 5×10⁸/mL, 2×10⁶/mL to 2×10⁸/mL, 5×10⁶/mL to 1×10⁸/mL, or 1×10⁷/mL to 5×10⁷/mL.

In one embodiment, the pharmaceutical composition may be a preparation for direct administration into the uterus of a subject or an injection for intravenous, intramuscular or subcutaneous administration, and preferably may be a preparation for direct administration into the uterus or an injection for subcutaneous administration.

In this case, the pharmaceutical composition may further comprise a pharmaceutically acceptable carrier. The pharmaceutically acceptable carrier may be any carrier that is a non-toxic material suitable for delivery to a patient. Distilled water, alcohol, fats, waxes, and inert solids may be included as carriers. Pharmacologically acceptable adjuvants (buffering agent and dispersing agent) may also be included in the pharmaceutical composition.

In another aspect of the present invention, there is provided a method of treating and/or preventing Asherman's syndrome or complications thereof, comprising administering mitochondria to a subject.

The mitochondria, Asherman's syndrome and complications thereof are as described above.

The administration may be a preparation for direct administration into the uterus or an injection for intravenous, intramuscular or subcutaneous administration, and preferably may be a preparation for direct administration into the uterus or a preparation for intravenous injection.

In order to ensure product stability according to the distribution of injection prescriptions, the pharmaceutical composition according to the present invention may be manufactured as a physically and chemically very stable injection by adjusting the pH using a buffer solution such as an aqueous acid solution or phosphate that may be used as an injection. The injection may further comprise a preservative, analgesic agent, solubilizer, or stabilizer.

Specifically, the pharmaceutical composition of the present invention may comprise water for injection. The water for injection is distilled water made to dissolve solid injections or to dilute water-soluble injections, and may be glucose injection, xylitol injection, D-mannitol injection, fructose injection, physiological saline, dextran 40 injection, dextran 70 injection, amino acid injection, Ringer's solution, lactic acid-Ringer's solution, or phosphate buffer solution in the pH range of 3.5 to 7.5, or sodium dihydrogen phosphate-citric acid buffer solution, etc.

The preferred dosage of the pharmaceutical composition may vary depending on the condition, body weight, gender, and age of the patient, the severity of the disease, and the route of administration, and may be administered once a day or in several divided doses. Specifically, the pharmaceutical composition may also be administered 1 to 10 times, 3 to 8 times, or 5 to 6 times.

The pharmaceutical composition may be administered to a subject diagnosed with Asherman's syndrome or complications thereof, or suffering from Asherman's syndrome or complications thereof. Specifically, the “subject” may be a subject with Asherman's syndrome and/or Asherman's syndrome complications whose symptoms may be improved by administration of the therapeutic composition according to the present invention, and the subject may have a uterus and may be a mammal. In one embodiment, the subject includes animals such as horses, sheep, pigs, goats, camels, antelopes, dogs, and the like, or humans. By administering the pharmaceutical composition to a subject, Asherman's syndrome and/or Asherman's syndrome complications may be effectively prevented and treated.

In addition, the pharmaceutical composition may further comprise known agents for preventing or treating Asherman's syndrome, agents for preventing or treating sterility, or agents for preventing or treating infertility.

In addition, administration of the pharmaceutical composition may be additionally combined with treatment for Asherman's syndrome, treatment for sterility, or treatment for infertility. The treatment for Asherman's syndrome may include resection through hysteroscopic surgery, installation of an intrauterine catheter, and estrogen or progesterone treatment.

In another aspect of the present invention, there is provided the use of mitochondria for the treatment of Asherman's syndrome or complications thereof.

In another aspect of the present invention, there is provided the use of mitochondria for the manufacture of a medicament for preventing or treating Asherman's syndrome or complications thereof.

In one example, it was confirmed that when a pharmaceutical composition comprising mitochondria as an active ingredient was injected directly into the uterus or intravascularly, intrauterine fibrosis was suppressed, and both the implantation rate and embryonic development were improved in an Asherman's syndrome mouse model. These results mean that the pharmaceutical composition of one embodiment may exhibit the effect of preventing, treating, or improving Asherman's syndrome and complications of Asherman's syndrome.

Hereinafter, the present invention will be described in more detail by way of the following examples. However, the following examples are only for illustrating the present invention, and the scope of the present invention is not limited thereto.

### Example 1. Obtainment of Umbilical Cord-Derived Mesenchymal Stem Cell Mitochondria

### Example 1.1. Culture of Umbilical Cord-Derived Stem Cells

Umbilical cord-derived mesenchymal stem cells (IRB number: No. 201411-BR-022-02 or No. 201806-BR-029-03) were obtained from Wharton's jelly of the umbilical cord and used in the experiment. The isolated umbilical cord-derived mesenchymal stem cells were cultured in Minimum Essential Medium Alpha Modification (MEM Alpha Modification, Hyclone) medium containing 10% fetal bovine serum (FBS; Gibco, Waltham, USA) and 1% penicillin/streptomycin antibiotics (P/S, Hyclone, Logan, USA) using a T-175 culture flask. The cells were maintained at 37° C. and 5% CO₂ conditions, and the next subculture was performed when the cell density reached approximately 80% to 90%.

### Example 1.2. Isolation of Umbilical Cord-Derived Mesenchymal Stem Cell Mitochondria

Mitochondria were isolated using the umbilical cord-derived mesenchymal stem cells cultured in Example 1.1 above. Based on 2×10⁷ cells, 400 μl of SHE buffer [0.25 M Sucrose, 20 mM HEPES (pH 7.4), 2 mM EGTA, 10 mM KCl, 1.5 mM MgCl₂, 0.1% defatted bovine serum albumin (BSA), pH 7.4] was added to suspend the cells, and then the cells were cultured at 4° C. for 5 minutes. The cell membrane was disrupted using a 1 ml syringe (Korea vaccine, Seoul, South Korea). In order to remove undisrupted cells and nuclei, centrifugation was performed at 1,500×g at 4° C. for 5 minutes. The supernatant was recovered, and then centrifugation was performed at 20,000×g at 4° C. for 10 minutes. In order to wash the isolated mitochondria, 2 ml of SHE buffer without BSA was added, and centrifugation was performed at 20,000×g at 4° C. for 10 minutes. The supernatant was removed, and then the mitochondria pellet was washed twice with Dulbecco's phosphate-buffered saline (DPBS; Welgene, South Korea). Thereafter, 200 μl of DPBS was added to suspend the mitochondria pellet and then stored at 4° C. to obtain umbilical cord-derived mesenchymal stem cell mitochondria. It was confirmed that the isolated mitochondria had the appearance of a light white suspension.

### Example 2. Obtainment of Mitochondria Derived from Human Peripheral Blood Mononuclear Cells and Plasma

### Example 2.1. Isolation and Culture of Human Peripheral Blood Mononuclear Cells

The donor's blood was transported in a heparin tube and used in the experiment. 15 ml to 25 ml of FICALL-PAQUE™ PLUS (GE Healthcare, Chicago, USA) was added to a Leucosep tube (Greiner bio-one, Kremsmunster, Austria), and centrifugation was performed at 1,500 rpm for 1 minute. Thereafter, the donor's blood was added at a volume of 1 to 2 times thereof onto the added Ficoll-Paque solution without mixing with Ficoll-Paque to form two density gradient layers. Thereafter, centrifugation was performed at 2,000 rpm for 20 minutes, and after centrifugation, it was confirmed that four density gradient layers were formed in the following order: plasma, peripheral blood mononuclear cells (PBMC), granulocytes containing Ficoll-paque (Ficoll-paque+granulocyte), and red blood cells (RBC). From the formed density gradient layers, plasma and peripheral blood mononuclear cells were isolated into new 50 ml tubes, respectively.

The recovered peripheral blood mononuclear cells were centrifuged at 1,200×g for 10 minutes, the supernatant was removed, then 5 ml of RBC lysis buffer (Biolegend, San Diego, USA) was added, and left to stand at 37° C. and 5% CO₂ for 5 minutes. An additional 45 ml of DPBS was added, and then centrifugation was performed at 1,200×g for 10 minutes. The supernatant was removed, and then 20 ml of DPBS was added, and then centrifugation was performed at 1.200×g for 10 minutes. Finally, the supernatant was removed, and then blood mononuclear cells obtained as a pellet were obtained. DPBS was added to the cells, and then the cells were suspended, and the cell number was measured.

Peripheral blood mononuclear cells isolated from human blood were cultured in RPMI-1640 (Hyclone, Logan, USA) medium containing 10% FBS and 1% P/S (Penicillin/Streptomycin) using a T-175 culture flask. The cells were maintained at 37° C. and 5% CO₂ conditions, and the next subculture was performed when the cell density reached approximately 80% to 90%.

### Example 2.2. Isolation of Human Peripheral Blood Mononuclear Cell Mitochondria

Mitochondria were obtained from human peripheral blood mononuclear cells in the same manner as in Example 1.2 above, except that mitochondria were used using human peripheral blood mononuclear cells cultured in Example 2.1.

### Example 2.3. Isolation of Human Plasma Derived Mitochondria

The plasma obtained in Example 2.1 was centrifuged at 25,000×g at 4° C. for 20 minutes to precipitate cell-derived substances present in the plasma, and then the supernatant was removed. Thereafter, mitochondria were obtained in the same manner using the SHE buffer used in Example 1.2.

### Example 3. Obtainment of Human-Derived Hepatocyte Mitochondria

### Example 3.1. Culture of Hepatocytes

WRL 68 (CL-48), a human-derived hepatocyte cell line, was purchased from ATCC and used in the experiment. WRL 68 was cultured in Dulbecco's Modified Eagle's Medium high glucose (DMEM; Hyclone) medium containing 10% FBS and 1% P/S using a T-175 culture flask. The cells were maintained at 37° C. and 5% CO₂ conditions, and the next subculture was performed when the cell density reached approximately 80% to 90%

### Example 3.2. Isolation of Human-Derived Hepatocyte Mitochondria

Mitochondria were obtained from human-derived hepatocytes in the same manner as in Example 1.2 above, except that mitochondria were used using human-derived hepatocytes cultured in Example 3.1.

### Example 4. Properties of Mesenchymal Stem Cell-Derived Mitochondria

### Example 4.1. Measurement of Protein in Isolated Mitochondria and Measurement of Size of Mitochondria

In order to measure the protein in the mitochondria isolated in Examples 1 to 3 above, the Bicinchoninic acid assay (BCA assay; Pierce, Rockford, USA) method was used. The concentration was measured in mitochondria suspended in 200 μl of DPBS according to the kit protocol using a 10 μl sample. The mitochondria content obtained from 2×10⁷ cells was calculated as protein concentration using the BSA standard curve.

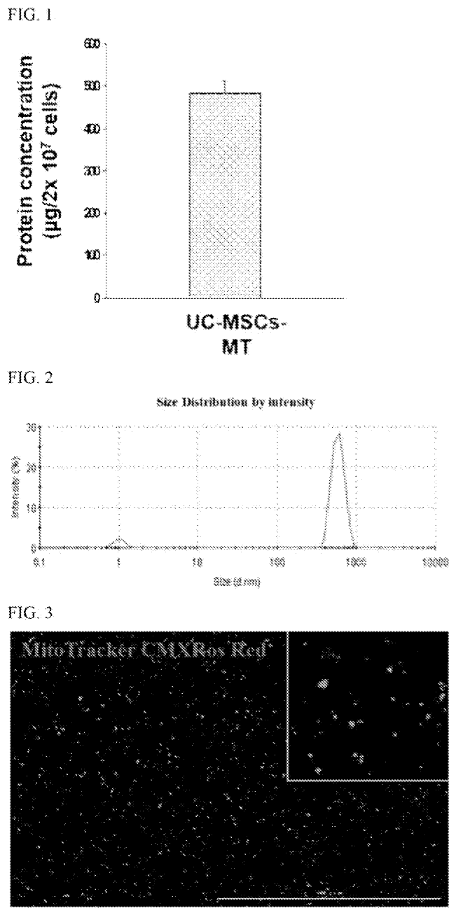

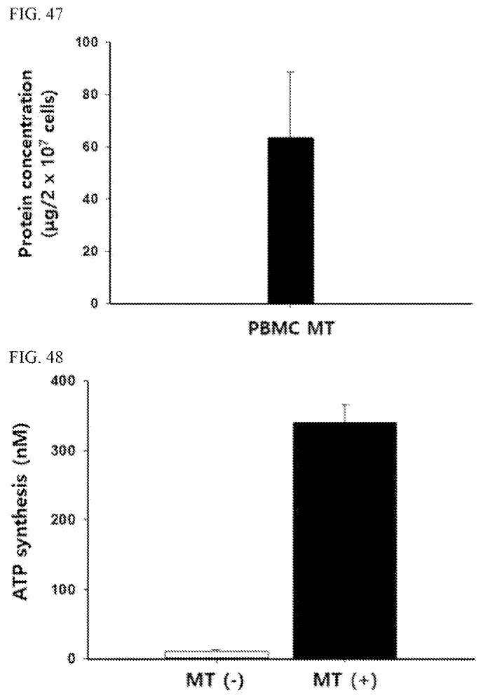

As a result, as shown in FIGS. 1, 46, and 47, it was confirmed that all of the umbilical cord-derived, hepatocyte-derived, and peripheral blood mononuclear cell-derived mesenchymal stem cells had sufficient mitochondrial protein content. In addition, as shown in FIG. 1, it was confirmed that the mitochondrial protein content of umbilical cord-derived mesenchymal stem cells was 484±28.3 μg.

In order to measure the size and distribution of the umbilical cord-derived mesenchymal stem cell mitochondria isolated in Example 1 above, they were analyzed using dynamic light scattering (DLS; Dynals, Protein solution Inc., Charlottesville, VA) equipment.

As a result, as shown in FIG. 2, it was confirmed that the size of the mitochondria was 650±108 nm.

### Example 4.2. Confirmation of Survival of Isolated Mitochondria

In order to confirm the umbilical cord-derived mesenchymal stem cell mitochondria isolated in Example 1 above, the mitochondria were stained with a mitochondrial membrane potential (MMP)-dependent MitoTracker CMXRos Red probe, and then fluorescence microscopy and flow cytometry were performed.

Specifically, the isolated mitochondria were each stained with Mitotracker CMXRos Red (Thermo Fisher, Waltham, USA; 300 nM), a mitochondria specific indicator, at 4° C. for 30 minutes. At this time, MitoTraker CMXRos Red is a marker that stains mitochondria in a mitochondrial membrane potential (MMP)-dependent manner. Confirmation of the marker is an experiment to confirm whether the isolated mitochondria maintain membrane potential and are viable. Next, the mitochondria were washed twice with DPBS, suspended in 200 μl of DPBS, and then the fluorescence signal was measured.

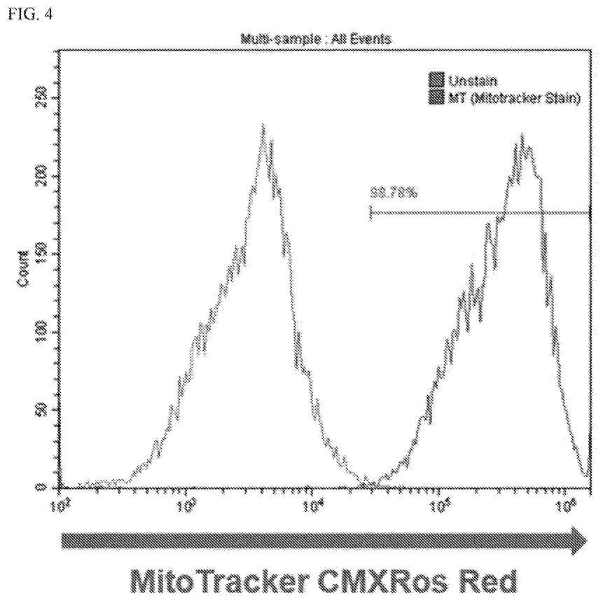

As a result, as shown in FIG. 3, it was confirmed that the isolated mitochondria bound to the Mito Tracker probe. In addition, as shown in FIG. 4, it was confirmed that the isolated mitochondria bound to the specific indicator (Mitotracker CMXRos Red).

### Example 4.3. Confirmation of Purity of Isolated Mitochondria

The purity of the umbilical cord-derived mesenchymal stem cell mitochondria isolated in Example 1 above was confirmed.

In order to confirm the purity of the isolated mitochondria, the presence of mitochondria specific markers [cytochrome C oxidase (COX IV), cytochrome C, Translocase of outer mitochondrial membrane 20 (TOMM20) and Apoptosis inducing factor (AIF)] and the absence of other cell organelle markers [KDEL (ER marker) and Proliferating Cell Nuclear Antigen (PCNA; nuclear marker)] were confirmed.

Specifically, in order to confirm the purity, the isolated mitochondria were heat-treated at 100° C. for 3 minutes using SDS-PAGE loading buffer (LPS solution, Daejeon, South Korea). The proteins were separated by size using a 12% SDS-PAGE gel and then transferred to a PVDF membrane at 0.35 mA for 120 minutes. The PVDF membrane to which the protein was transferred was blocked with TBS-T [Water, 150 mM NaCl, 10 mM Tris-HCl, 0.1% (v/v) Tween-20, pH 7.6] containing 3% BSA for 90 minutes at room temperature. After blocking, without removing the buffer, the primary antibodies were treated with KDEL (Invitrogen, PA1-013), PCNA (Santa Cruz Biotechnology, sc-56), cytochrome C (Santa Cruz Biotechnology, sc-13156), COX IV (Abcam, ab33985). TOMM20 (Santa Cruz Biotechnology, sc-17764), and AIF (Santa Cruz Biotechnology, sc-13116) at a ratio of 1:1,000 and reacted at 4° C. overnight.

As a result, as shown in FIG. 5, the presence of mitochondrial proteins was confirmed in all mitochondria specific markers (COX IV, cytochrome C, TOMM20, AIF) in the mitochondria fraction, but other cell organelle markers KDEL and PCNA were confirmed not to be present. At this time, in FIG. 5, M represents the fraction containing mitochondria, and C represents the cell fraction without mitochondria.

### Example 4.4. Confirmation of Activity of Isolated Mitochondria

The activity of the umbilical cord-derived mesenchymal stem cell mitochondria isolated in Example 1 above was confirmed. Specifically, the ATP content. ROS production, membrane potential, and ATP synthesis ability of mitochondria were confirmed.

### Example 4.4.1. Confirmation of ATP Content Included in Mitochondria

In order to confirm the ATP content, an experiment for the isolated mitochondria was performed using the CellTiter-Glo luminescence assay kit (Promega, Madison, WI). DPBS (MT (−)) and 10 μg of mitochondria suspended in 100 μl of DPBS (MT(+)) were each dispensed into a white 96-well plate, and 100 μl of CellTiter-Glo reagent was added according to the kit protocol using a sample. After mixing on a shaker for 2 minutes, the light was blocked and allowed to react for 10 minutes. The luminescence values were measured using a luminescence microplate reader (Epoch Spectrometer, BioTek Inc.).

### Example 4.4.2. Confirmation of ROS Production in Mitochondria

In order to confirm the ROS production in mitochondria, mitochondria ROS(mROS) was measured using MitoSOX Red (Invitrogen, Carlsbad, CA), a mitochondrial superoxide indicator. The MT(+) group containing the isolated mitochondria and the MT(−) group containing the same volume of PBS were dispensed into a 96-well black plate, and then treated with 1 μM of MitoSOX Red, and allowed to react at 37° C. and 5% CO₂ for 30 minutes. The fluorescence intensity was measured at an absorption wavelength of 510 nm/emission wavelength of 528 nm using a fluorescence microplate reader (BioTek Inc.).

### Example 4.4.3. Confirmation of Mitochondrial Membrane Potential

In order to confirm the mitochondrial membrane potential, the mitochondrial membrane potential was measured using JC-1 (Invitrogen). The MT(+) group containing the isolated mitochondria, the MT(−) group consisting of the same volume of PBS alone, and the MT(+)+CCCP group containing the isolated mitochondria treated with CCCP (carbonyl cyanide m-chlorophenyl hydrazone, Sigma Aldrich) were added to a 96-well black plate, and treated with 1 μM JC-1 dye, and allowed to react at 37° C. and 5% CO₂ for 30 minutes. JC-1 accumulated in mitochondria depending on the membrane potential (MMP), changing the fluorescence value from the green emission wavelength range (absorption 485 nm/emission 516 nm) to red (absorption 579 nm/emission 599 nm). MMP was determined as a ratio of fluorescence values, which was measured using a fluorescence microplate reader.

### Example 4.4.4. Confirmation of ATP Synthesis Ability of Mitochondria

In order to confirm the ATP synthesis ability, the mitochondria were divided into an intact mitochondria (intact MT) group and a damaged mitochondria (damaged MT) group.

Specifically, the damaged mitochondria (damaged MT or dead MT) were prepared by treating them with 50 μM CCCP (positive control group as mitochondrial oxidative phosphorylation uncoupler). The mitochondria prepared as described above were each suspended in 100 μl of DPBS, and 10 μg of mitochondria were prepared in a white 96-well plate, and 5 mM ADP was added, and then reacted in an incubator at 37° C. After 45 minutes, 100 μl of CellTiter-Glo reagent was added and mixed on a shaker for 2 minutes, and then the light was blocked and allowed to react for 10 minutes. The luminescence values were measured using a luminescence microplate reader.

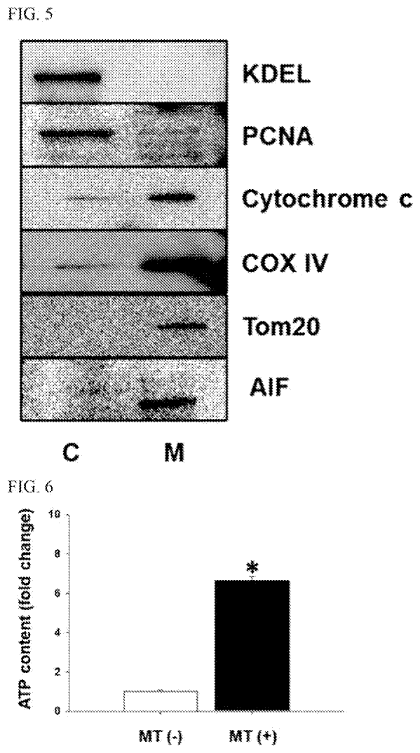

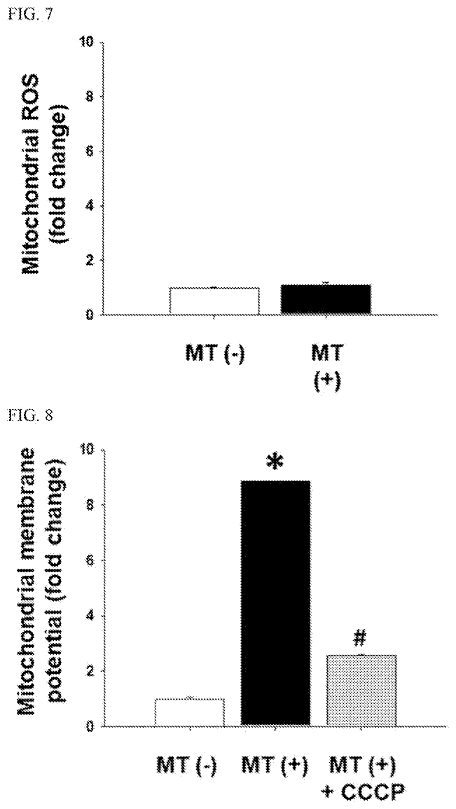

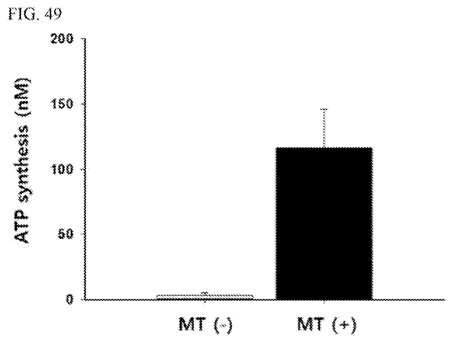

As a result, as shown in FIGS. 6, 48, and 49, it was confirmed that the ATP content was measured to be high in the isolated mitochondria compared to the state in which no mitochondria were present [MT(−)]. In addition, as shown in FIG. 7, it was confirmed that the activity of ROS in mitochondria was low.

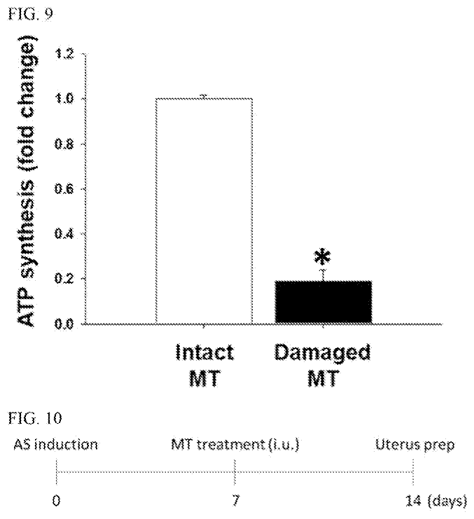

In addition, as shown in FIG. 8, the membrane potential was confirmed in the mitochondria group. In addition, as shown in FIG. 9, the ATP synthesis ability of the isolated mitochondria was confirmed. In addition, a decrease in membrane potential and ATP synthesis ability were confirmed due to loss of mitochondrial function by treatment with CCCP. These results mean that the isolated mitochondria of the present invention maintain the activity of mitochondria.

### Example 5. Analysis of Effects on Uterine Regeneration and Fibrosis Reduction by Isolated Mitochondria

### Example 5.1. Construction of Asherman's Syndrome Mouse Model

An Asherman's syndrome mouse model was constructed using 8-week-old female mice. This study was conducted with approval from the Institutional Animal Care and Use Committee (IACUC, approval number 200159). In accordance with institutional guidelines for experimental animals, mice were maintained under temperature and light controlled conditions for 12 hours daily at the Laboratory Animal Center of CHA University. After administering an anesthetic (Avertin) to the mouse by intraperitoneal injection, the mouse outer/inner skin were vertically incised and the uterus was exposed. Next, a small incision was made in the uterus located at the fallopian tube junction in the mouse, and then a 26-gauge needle was inserted into the uterus and rotated to induce trauma, and then recovered to obtain an Asherman's syndrome mouse model.

### Example 5.2. Analysis of Changes in Fibrosis Indicators

In order to confirm the histological improvement effect by administration of the isolated mitochondria, changes in fibrosis indicators by administration of the isolated mitochondria were analyzed.

Specifically, as shown in FIG. 10, on the 7th day after the construction of the Asherman mouse model, 10 μg of the mesenchymal stem cell-derived mitochondria in Example 1 above was administered by direct delivery into the endometrium of the mouse model, and then the uterus was obtained on the 14th day.

Next, immunostaining was performed for histological analysis. The tissue was fixed in a fixative solution for one week, and then a block was constructed through an infiltration process in a paraffin solution. This was cut into 5 μm thin sections and attached to a slide, and then staining was performed. For molecular biological analysis, in order to analyze the expression level of fibrosis-related factors Col1a1, Col3a1, Timp1, and Tgfβ1, real-time RT-PCR was performed for quantification. RNA was isolated from the tissue using Trizol, and then cDNA was synthesized. In order to analyze the expression of mRNA, primers were designed and PCR was performed. The primer sequences used in the experiment are as follows.

Table 1 below shows the primer sequences used in the experiment.

TABLE 1

| Gene | | Sequence (5′→3′) | Size (bp) |
|---|---|---|---|
| Col1a1 | Forward | CTGGCGGTTCAGGTCCAAT (SEQ ID NO: 1) | 141 |
| | Reverse | TTCCAGGCAATCCACGAGC (SEQ ID NO: 2) | |
| Col3a1 | Forward | ACGTAGATGAATTGGGATGCAG (SEQ ID NO: 3) | 154 |
| | Reverse | GGGTTGGGGCAGTCTAGTGGC (SEQ ID NO: 4) | |
| Timp1 | Forward | GGGTTCCCCAGAAATCAACGAG (SEQ ID NO: 5) | 139 |
| | Reverse | ACAGAGGCTTTCCATGACTGGGGTG (SEQ ID NO: 6) | |
| Tgfβ1 | Forward | GTGAAACGGAAGCGCATCGAAG (SEQ ID NO: 7) | 193 |
| | Reverse | CATAGTAGTCCGCTTCGGGCTCC (SEQ ID NO: 8) | |

As a result, as shown in FIG. 11, it was confirmed that the accumulation of COL1A1 (collagen), which appears blue by Masson's trichrome staining, was increased in the Asherman's syndrome uterine group (AS), but was reduced by MT treatment. These results mean that fibrotic lesions were reduced by MT treatment.

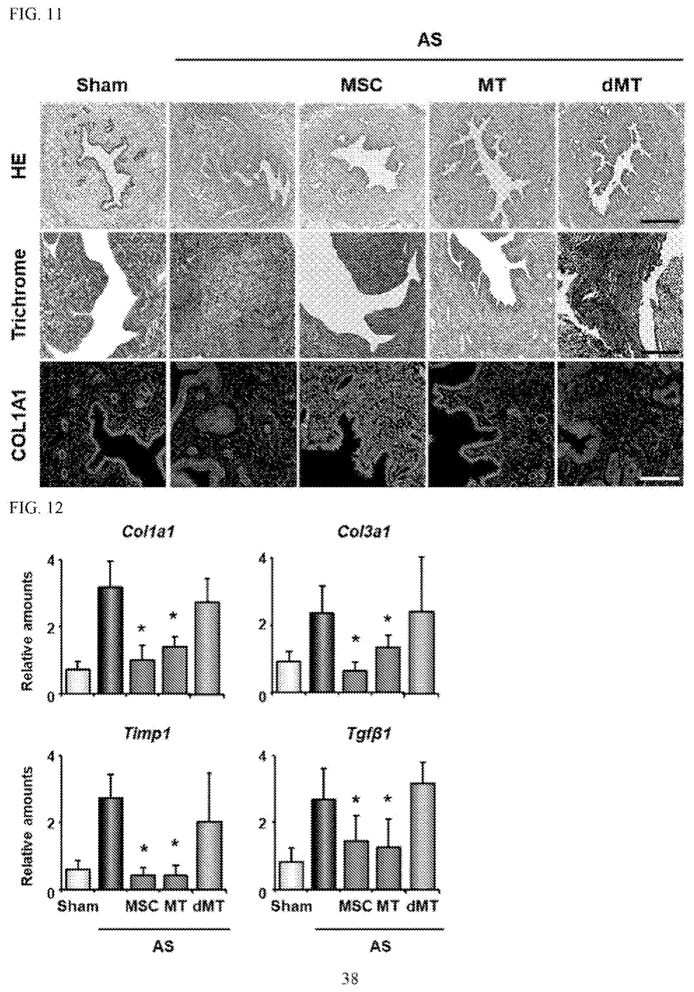

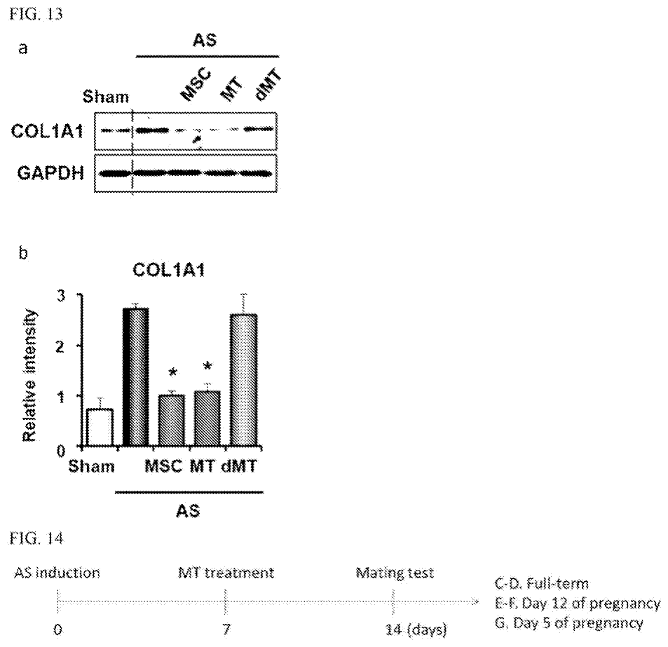

In addition, as shown in FIGS. 12 and 13, it was confirmed that the expression of fibrosis factors (Col1a1, Col3a1, Timp1, and Tgfβ1) at the mRNA level and protein level is increased in the AS group, and the expression is reduced in the MT treatment group similarly to the MSC treatment group. These results mean that the fibrosis phenotype of Asherman's syndrome was significantly reduced in the uterus of the Asherman's syndrome mouse model injected with MT.

### Example 6. Analysis of Functional Improvement Effect by Administration of Isolated Mitochondria

### Example 6.1. Analysis of Implantation Rate, Delivery Rate, and Litter Size by Administration of Isolated Mitochondria

In order to confirm the functional improvement effect by administration of the isolated mitochondria, an experiment was performed in a manner as shown in FIG. 14. Specifically, on the 7th day after the induction of the mouse model of Example 5.1 above, 10 μg of the mitochondria in Example 1 above was administered by direct delivery into the endometrium.

Next, on the 7th day after administration, the mice were housed together for mating with male mice, and mating was confirmed by checking the plug observed in the female genital tract every morning after normal mating. When the day the plug was confirmed was set as day 1, the number and weight of implanted embryos were observed on the 12th day of pregnancy, which corresponds to the second trimester of pregnancy.

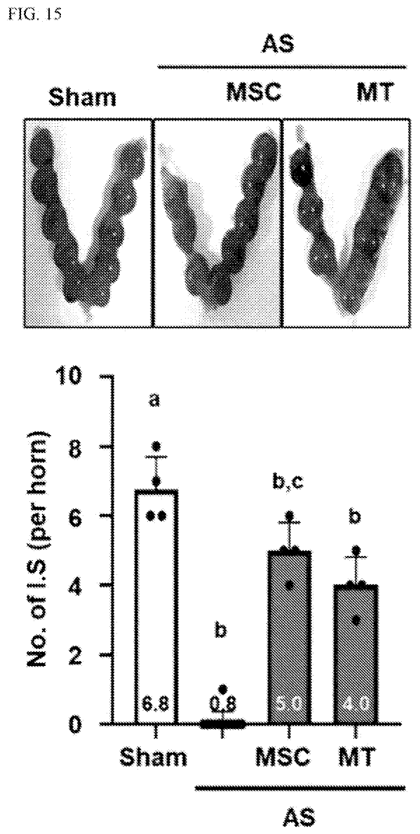

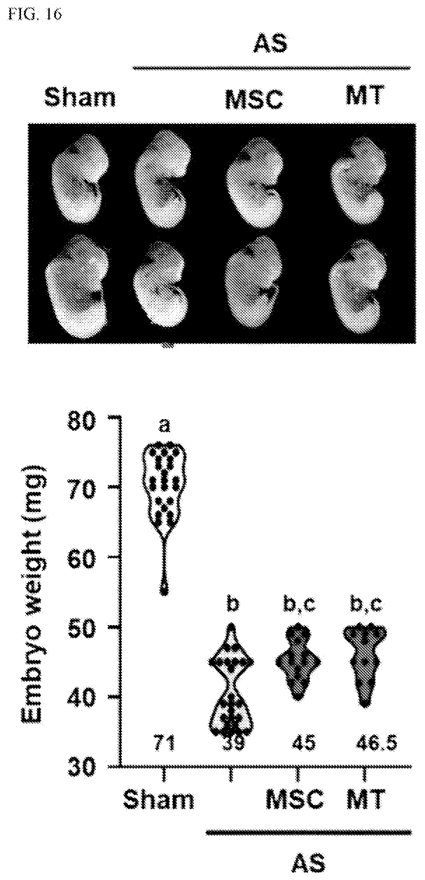

As a result, as shown in FIG. 15, it was confirmed that the number of implanted embryos was increased with the administration of the isolated mitochondria (MT) through the 12th day of pregnancy in the mouse model, which represents the second trimester of pregnancy in humans. In addition, as shown in FIG. 16, only the implanted embryos were obtained and the weights were measured. As a result, the weight was also increased compared to the Asherman's syndrome (AS) group.

### Example 6.2. Analysis of Embryonic Development by Administration of Isolated Mitochondria

In order to confirm the degree of embryonic development at the end of pregnancy by the administration of isolated mitochondria, the mice were sacrificed using CO₂ on the morning of the 12th day of pregnancy, and the uterus was completely exposed by making a vertical incision in the outer/inner skin from the ventral side. Next, the number of embryos implanted in the exposed uterus was determined and diagrammed.

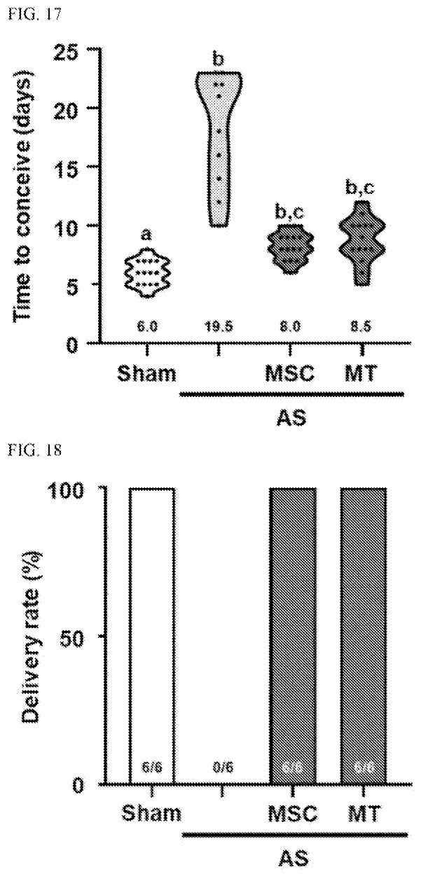

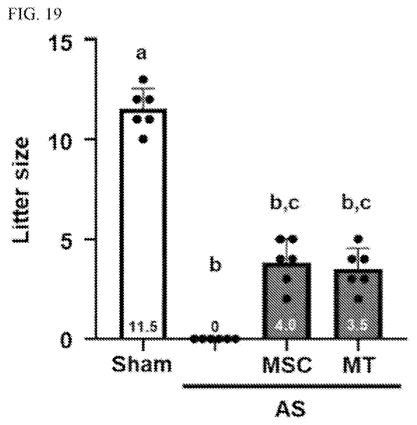

As a result, as shown in FIG. 17, it was confirmed that the AS group had a relatively long time to conceive, similar to the irregular reproductive cycle observed in actual Asherman's syndrome patients, but the MT group had a shortened time to conceive similar to the normal group (Sham). In addition, as shown in FIGS. 18 and 19, the MT group had improved delivery rate and litter size similar to the MSC group.

### Example 7. Analysis of Early Embryo Implantation Rate by Administration of Isolated Mitochondria

The number of embryos implanted in the early stage of pregnancy in the Asherman mouse model treated in the same manner as in Example 6.1 above was confirmed. Specifically, on the 5th day of pregnancy, which corresponds to the early stage of pregnancy, a Chicago blue solution was intravenously injected into the mouse model to increase the permeability of blood vessels around the embryo implantation, thereby confirming the stained implantation site.

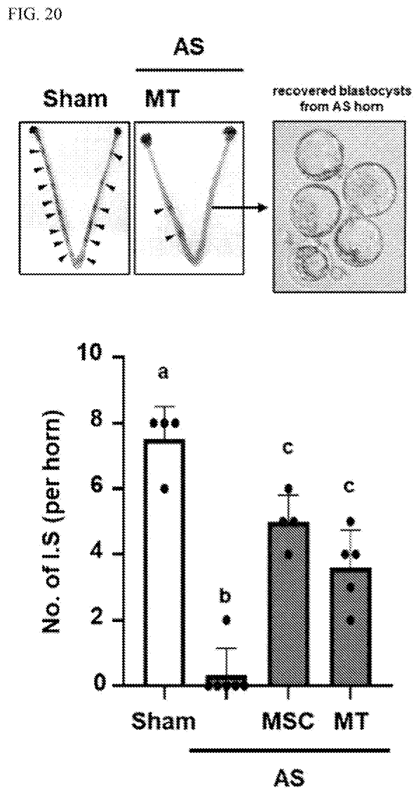

As a result, as shown in FIG. 20, in the AS group, an embryo that failed to implant was confirmed in the uterus. However, in the MT group, implantation of the embryo occurred at a normal time, and in the MT group, the number of implanted embryos was increased on the 5th day of pregnancy.

### Example 8. Analysis of Cell Proliferation Effect by Administration of Isolated Mitochondria

### Example 8.1. Immunofluorescence Staining Analysis of Vascular Endothelial Cell Markers

In order to confirm the cell proliferation effect by administration of the isolated mitochondria, the mesenchymal stem cell-derived mitochondria of Example 1 above were administered to the uterus of the mouse model of Example 5.1 above, and immunofluorescence staining was performed. Specifically, in the uterus of the mouse model, blood vessels were stained using the vascular endothelial cell marker CD31, and proliferating cells were stained using the cell proliferation marker KI-67.

The method for obtaining samples required for staining was as follows. The uterus administered with mitochondria was extracted and fixed in a fixative solution, and then an infiltration process was performed, and a paraffin block was constructed. 5 μm thin sections were attached to a slide using a paraffin block cutter, and then staining was performed. The stained sections were observed and photographed using a fluorescence microscope, and multiple people used the same photograph to count the number of total cells and the number of cells stained with each antibody, which were then graphed.

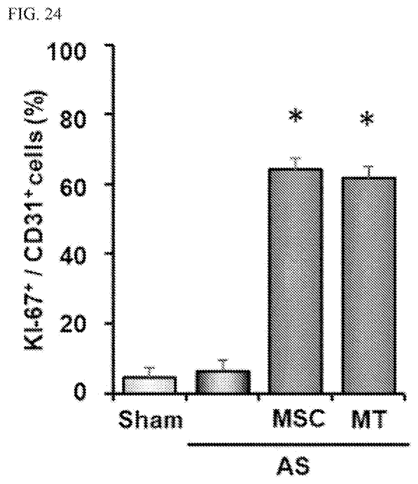

As a result, as shown in FIGS. 23 and 24, KI-67⁺ proliferating cells among CD31⁺ vascular cells were counted, and the number was summarized as a percentage, resulting in about 60% of all vascular cells. Therefore, it was confirmed that the ratio of the proliferating vascular cells was high. These results mean that when isolated mitochondria are administered, vascular endothelial cells of the endometrium proliferate effectively.

### Example 8.2. Confirmation of Vascular Endothelial Cell Markers

In order to confirm the cell proliferation effect by administration of the isolated mitochondria, the expression of vascular endothelial cell markers in the uterus of the mouse model obtained under the same conditions as in Example 5.1 above was confirmed at the miRNA level.

Table 2 below shows the primer sequences used in the experiment.

TABLE 2

| Gene | | Sequence (5′→3′) | Size (bp) |
|---|---|---|---|
| Hgf | Forward | CTGACCCAAACATCCGAGTTG (SEQ ID NO: 9) | 125 |
| | Reverse | TTCCCATTGCCACGATAACAA (SEQ ID NO: 10) | |
| Igf1 | Forward | TGCTTCCGGAGCTGTGATCT (SEQ ID NO: 11) | 125 |
| | Reverse | CGGGCTGCTTTTGTAGGCT (SEQ ID NO: 12) | |
| Ang1 | Forward | GGGACAGCAGGCAAACAGA (SEQ ID NO: 13) | 110 |
| | Reverse | TGTCGTTATCAGCATCCTTCGT (SEQ ID NO: 14) | |
| Vegfa | Forward | GCAGGCTGCTGTAACGATGA (SEQ ID NO: 15) | 105 |
| | Reverse | GCATGATCTGCATGGTGATGTT (SEQ ID NO: 16) | |
| Hif1α | Forward | ACAAGTCACCACAGGACAG (SEQ ID NO: 17) | 168 |
| | Reverse | AGGGAGAAAATCAAGTCG (SEQ ID NO: 18) | |
| Hif2α | Forward | AATGACAGCTGACAAGGAGAAAAA (SEQ ID NO: 19) | 257 |
| | Reverse | GAGTGAAGTCAAAGATGCTGTGTC (SEQ ID NO: 20) | |

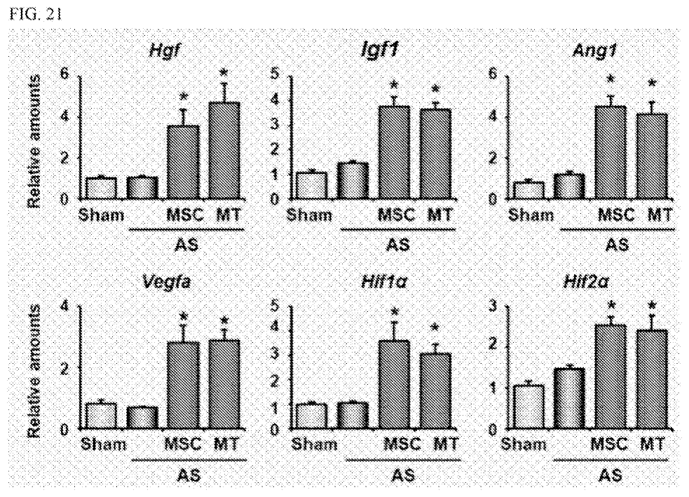

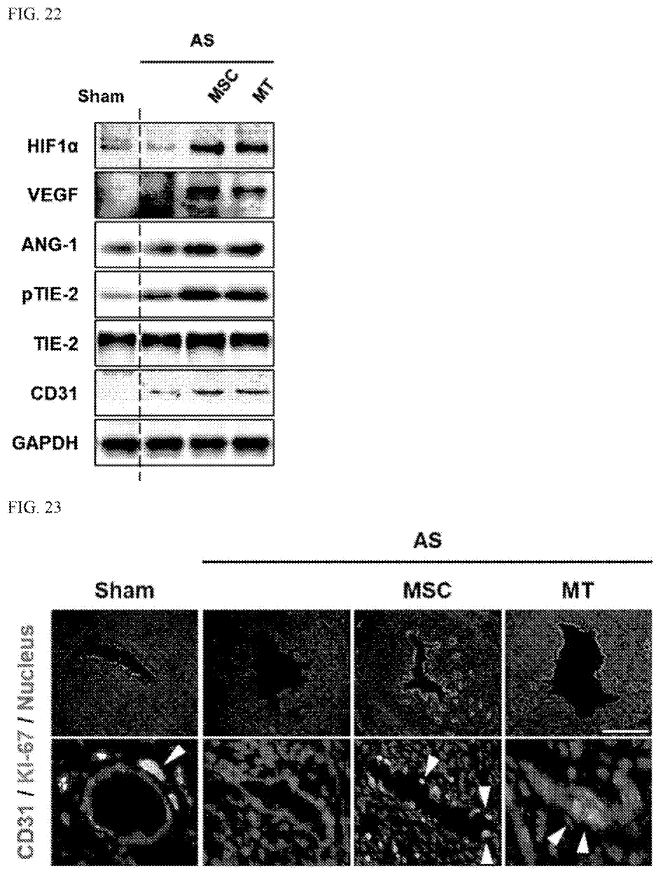

As a result, as shown in FIGS. 21 and 22, the mRNA expression of Hgf, Igf1, Ang1, Vegfa, Hif1α, and Hif2α, known as vascular endothelial cell markers, was significantly increased in the MT group compared to the AS group, and the degree of increase was similar to the MSC group.

### Example 8.3 Analysis of Changes in Fibrosis Indicators by Administration of Dead MT

In order to confirm the difference in changes in fibrosis indicators depending on the activity of isolated mitochondria, artificial death of stem cell-derived MTs was induced in the same manner as in Example 4.4.4 above and then injected into the mouse model of Example 5.1 above. Next, the expression of fibrosis-related factors Col1a1, Col3a1, Timp1, and Tgfβ1 was analyzed through RT-PCR and real-time RT-PCR in the same manner as in Example 5.2 above.

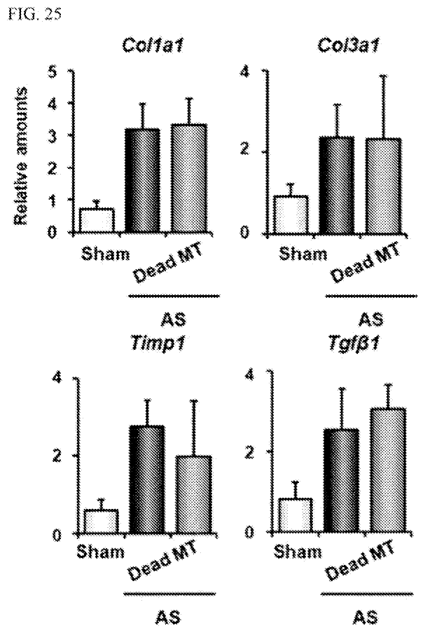

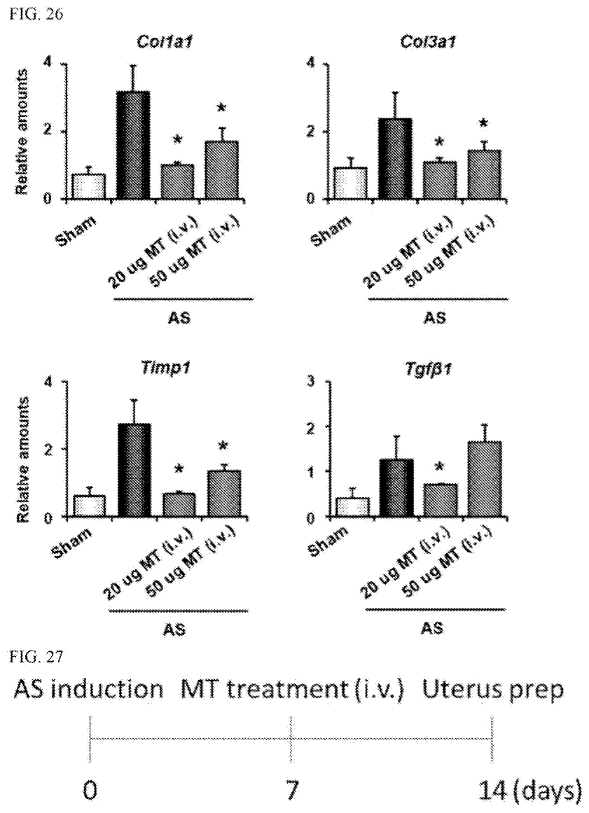

As a result, as shown in FIG. 25, when dead MT was injected, the fibrosis status was not reduced at all, but when live MT was injected, the fibrosis status was reduced. In addition, as shown in FIG. 26, it was confirmed that there was the fibrosis improvement effect depending on the injection dose. The injection dose was determined by quantifying the mitochondrial protein content as in Example 4.1.

### Example 9. Analysis of Changes in Fibrosis Indicators by Administration Method and Concentration of Isolated Mitochondria

In order to confirm the difference in changes in fibrosis indicators by the administration method and concentration of the isolated mitochondria, as shown in FIG. 27, the stem cell-derived mitochondria were injected to the mouse model of Example 5.1 above by intravenous administration.

Specifically, as shown in FIG. 27, on the 7th day after the construction of the Asherman mouse model. 10 μg of the stem cell-derived mitochondria was administered by injection into the vein located in the tail of the mouse model, and then the uterus was obtained on the 14th day.

Next, immunostaining was performed for histological analysis. The tissue was fixed in a fixative solution for one week, and then a block was constructed through an infiltration process in a paraffin solution. This was cut into 5 μm thin sections and attached to a slide, and then staining was performed. For molecular biological analysis, in order to analyze the expression level of fibrosis-related factors Col1a1, Col3a1, Timp1, and Tgfβ1, real-time RT-PCR was performed for quantification. RNA was isolated from the tissue using Trizol, and then cDNA was synthesized. In order to analyze the expression of mRNA, primers were designed and PCR was performed.

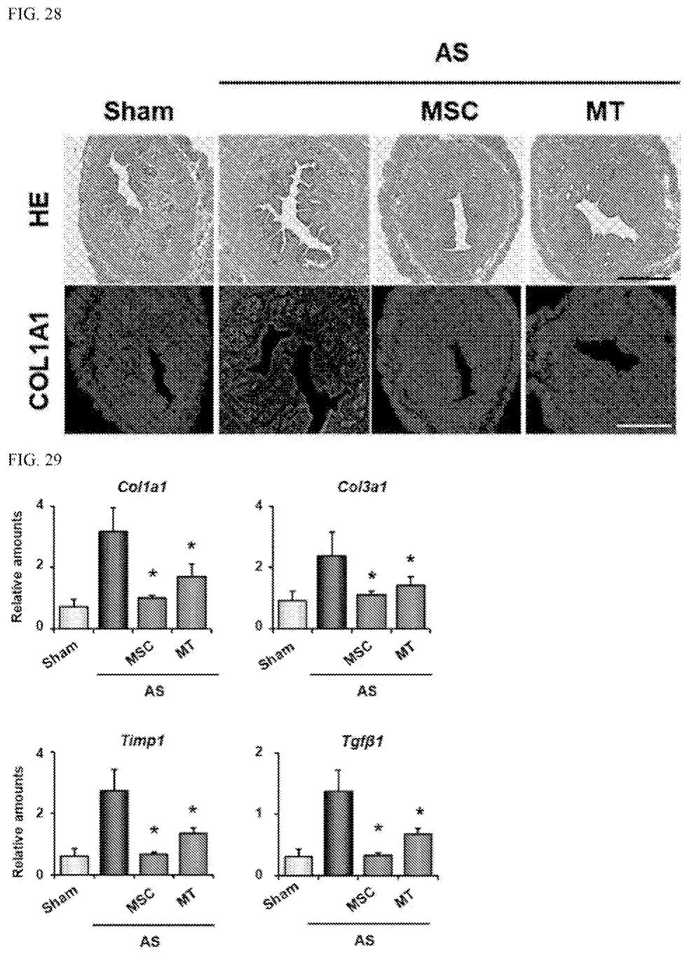

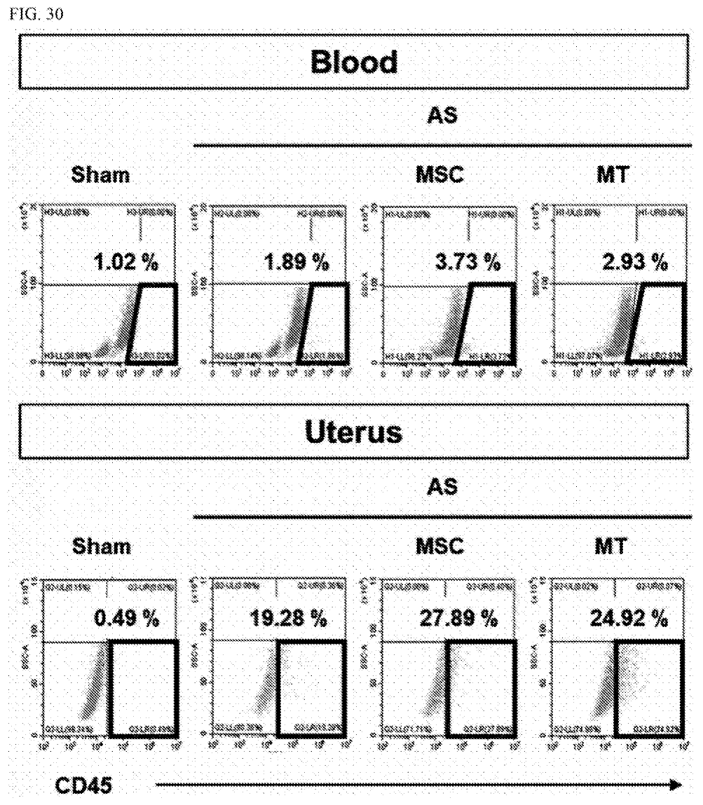

As a result, as shown in FIG. 28, even when the delivery method of the isolated mitochondria was changed to intravenous administration, it was confirmed that the expression of COL1A1 was reduced by MT injection, as confirmed through immunostaining. In addition, as shown in FIG. 29, the results observed at the mRNA level confirmed that the expression of Col1a1, Col3a1, Timp1, and Tgfβ1 was lowered in the MT group, indicating that it has the effect of improving fibrosis. These results mean that both the method of directly injecting isolated mitochondria into damaged tissue and the method of injecting them intravenously have the effect of improving fibrosis by mitochondria.

In addition, changes in immune cells in mice by intravenous administration of isolated mitochondria were confirmed. Specifically, immune cells from the blood and uterus of mice were isolated, and whether there was difference in quantitative changes in immune cells was observed through flow cytometry.

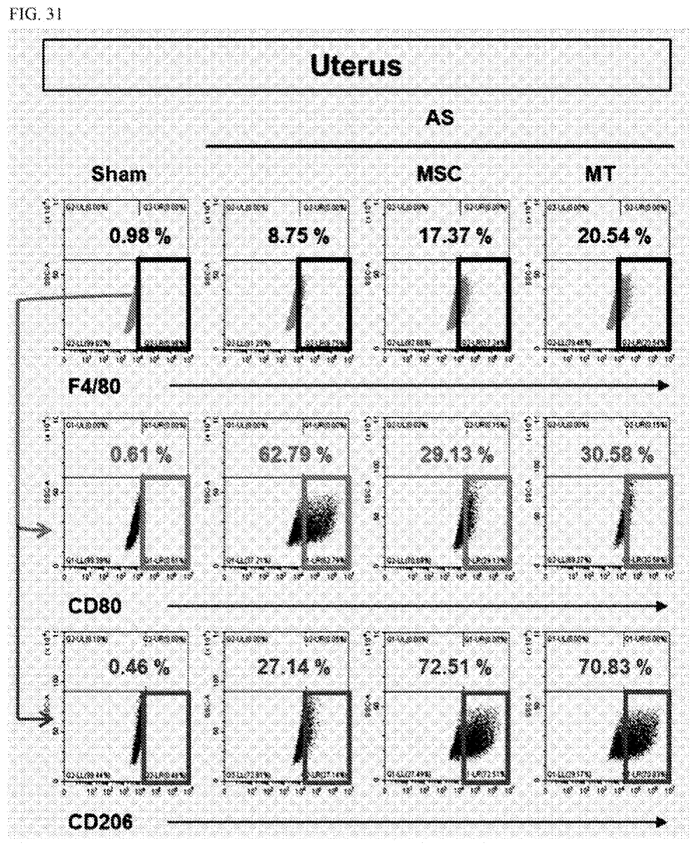

As a result, as shown in FIGS. 30 and 31, various changes were observed in the immune cells of the blood and uterus of AS mice administered with mitochondria. In particular, as shown in FIG. 30, the infiltration of total immune cells expressing CD45 was significantly observed in the uterus. In addition, as shown in FIG. 31, it was confirmed that the expression of F4/80, a macrophage marker, was increased in mice administered with mitochondria, the expression of CD80, an inflammatory marker, was reduced similarly to the group administered with MSC, and the expression of CD206, an anti-inflammatory marker, was increased similarly to the group administered with MSC.

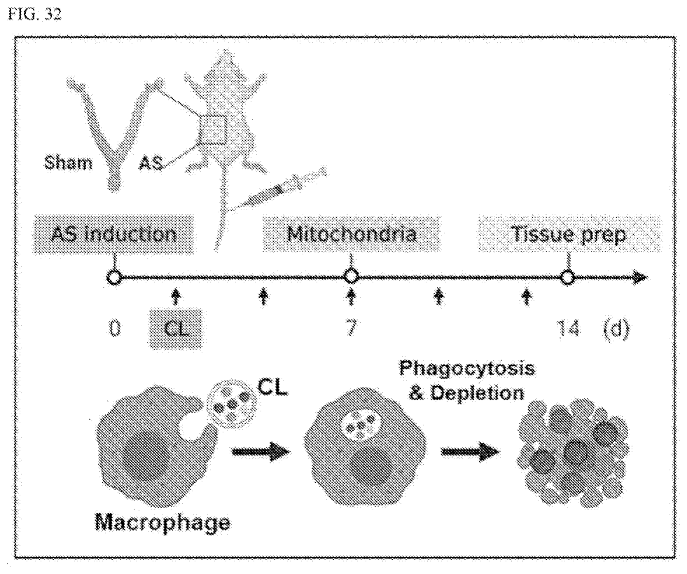

Next, in order to confirm the importance of macrophages in the process of regenerating the damaged uterus following the administration of mitochondria, as shown in FIG. 32, liposomes containing a toxic substance called clodronate (CL) were administered via an intravenous injection to create a macrophage-deficient environment in the mouse model of Example 5.1 above.

On the 7th day after the construction of the Asherman mouse model, 10 μg of the stem cell-derived mitochondria was administered by injection into the vein located in the tail of the mouse model, and then the uterus was obtained on the 14th day.

Next, immunostaining was performed for histological analysis. Specifically, the tissue was fixed in a fixative solution for one week, and then a block was constructed through an infiltration process in a paraffin solution. This was cut into 5 μm thin sections and attached to a slide, and then staining was performed. In addition, for molecular biological analysis, in order to analyze the expression level of fibrosis-related factors Col1a1 and Col3a1, real-time RT-PCR was performed for quantification. RNA was isolated from the tissue using Trizol, and then cDNA was synthesized. In order to analyze the expression of mRNA, primers were designed and PCR was performed

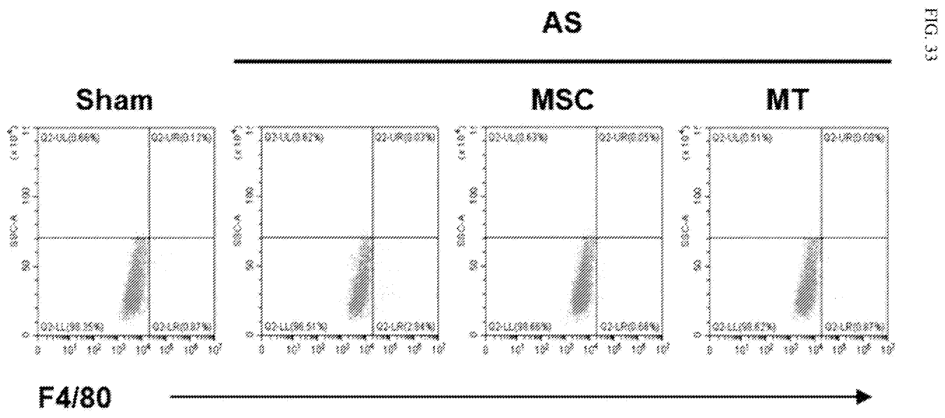

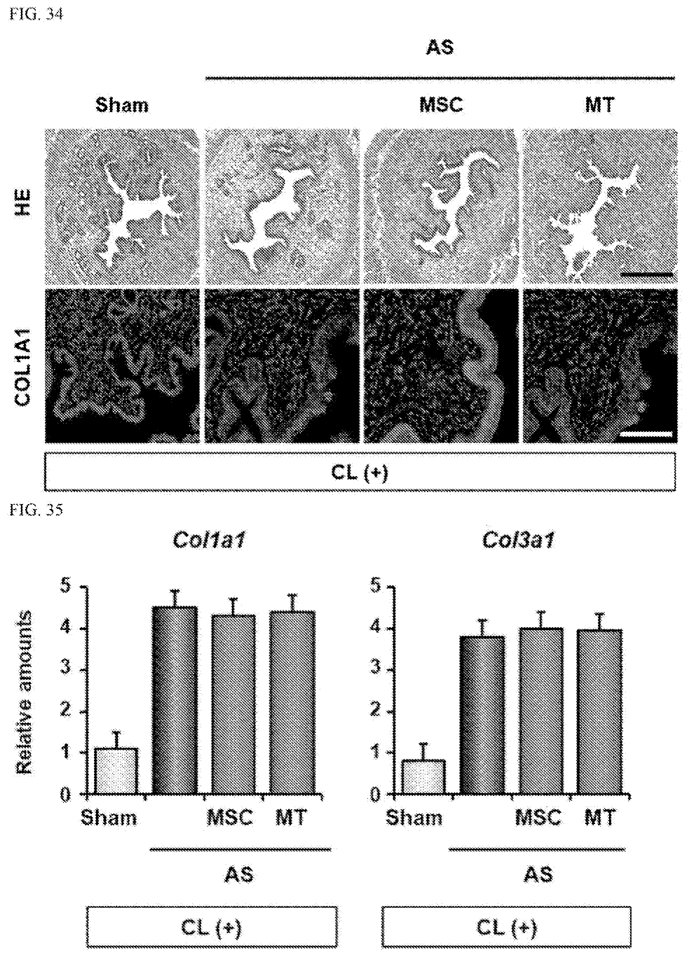

As a result, as shown in FIG. 33, the expression of F4/80, a macrophage marker, was not confirmed, indicating that all groups were deficient in macrophages in the uterus. In addition, as shown in FIGS. 34 and 35, it was confirmed through COL1A1 fluorescent immunostaining and real-time RT-PCR that tissue regeneration did not occur even when MT was injected in a macrophage-deficient state. These results mean that the control of fibrosis by administration of mitochondrial is highly related to macrophages.

### Example 10. Confirmation of Macrophage Polarization Regulation of Isolated Mitochondria

Using RAW264.7, a mouse macrophage cell line, it was confirmed whether isolated mitochondria affect the polarization of macrophages.

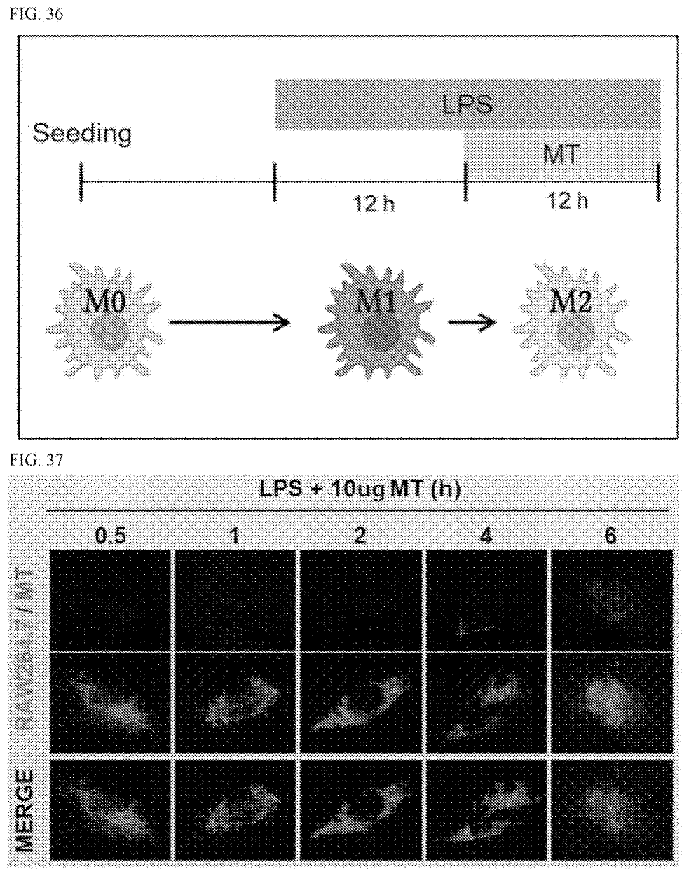

Specifically, as shown in FIG. 36, an M1 polarization state, which is an inflammatory environment, was created using LPS, and an M2 polarized environment in which the inflammatory environment was alleviated was created by treating with 10 μg of the isolated mitochondria (quantification based on mitochondrial protein).

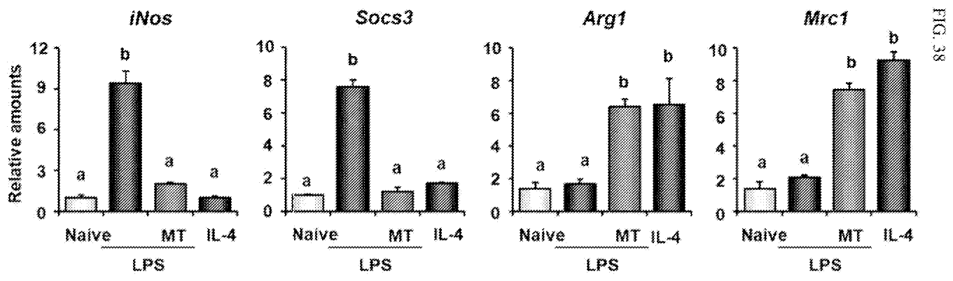

As a result, as shown in FIG. 37, it was confirmed that the treated mitochondria (red) were located within macrophages (green) within 4 to 6 hours. The mitochondria were used to identify their location by staining them in different colors using Mito tracker. In addition, as shown in FIG. 38, it was confirmed at the mRNA level that the expression of inflammatory factors (INOS and Socs3) was reduced in the MT treatment group, and the expression of anti-inflammatory factors (Arg1 and Mrc1) was increased in the MT treatment group.

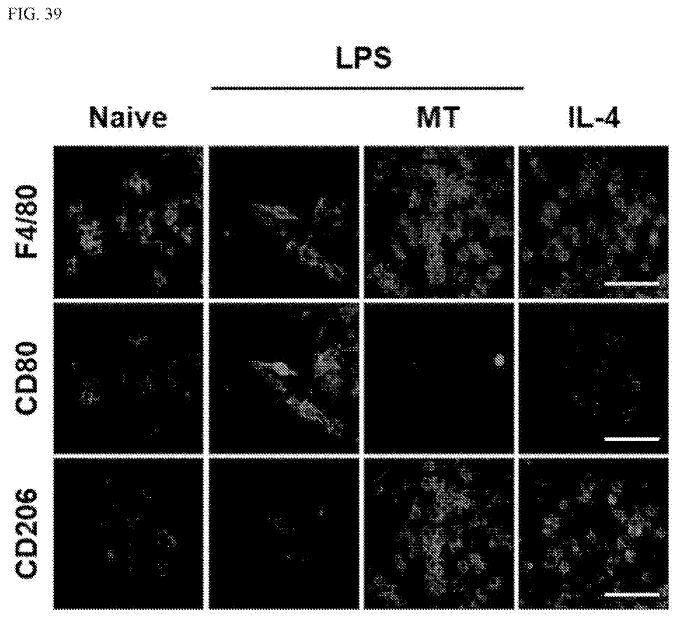

In addition, the inflammatory marker CD80 was reduced and the anti-inflammatory marker CD206 was increased in the MT treatment group, similar to the group in which M2 polarization of macrophages was induced by IL-4 treatment. At this time, the cells were treated with IL-4 at a concentration of 10 ng/ml for 12 hours. The cells treated with the material were fixed using a fixative solution, and blocking was performed with 4% BSA at room temperature for 1 hour. Thereafter, CD80 and CD206 were each diluted 1:200 in 4% BSA and reacted with the cells. It was refrigerated at 4° C. for one day, and each secondary antibody was diluted 1:1000 in 4% BSA and reacted at room temperature for 1 hour. Thereafter, mounting was performed and the image in FIG. 39 was obtained using a fluorescence microscope. Finally, the same results were confirmed through FACS experiment.

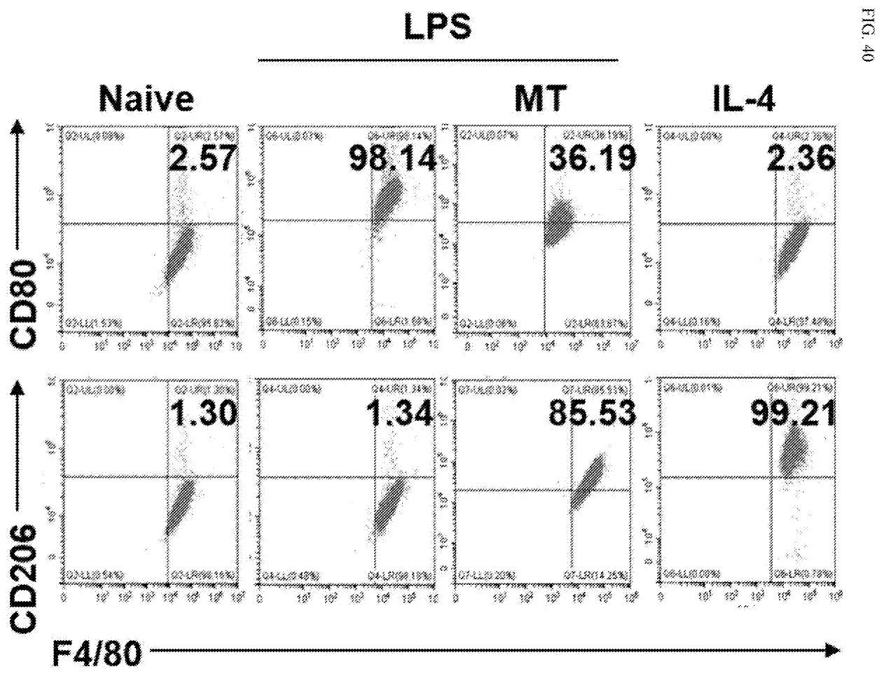

The cells treated with the material were collected in a tube using trypsin, and then each fluorescent-attached antibody was diluted 1:200 in FACS buffer (DPBS+0.2% BSA) and reacted with the cells. After 30 minutes, washing was performed twice using FACS buffer, and then the number of cells reacting with antibodies was analyzed using a FACS device, as shown in FIG. 40. These results mean that when macrophages were treated with isolated mitochondria, the expression of CD80 was reduced and the expression of CD206 was increased, indicating that the mitochondria polarized the macrophages from M1 to M2.

### Example 11. Confirmation of Promotion of HUVEC Migration and Formation of Isolated Mitochondria

Using the method of Example 10, it was confirmed whether the isolated mitochondria of Example 1 affect the migration and tube formation of human umbilical vein endothelial cells (HUVEC).

Specifically, as shown in FIG. 41, macrophages whose polarization was induced by MT treatment were co-cultured with HUVEC cells to observe the migration and tube formation of HUVEC cells. At this time, in order to observe the migration of HUVEC cells, space was created at regular intervals, and then co-culture was performed. At this time, in the control group, M2 polarization of macrophages was induced by IL-4 treatment.

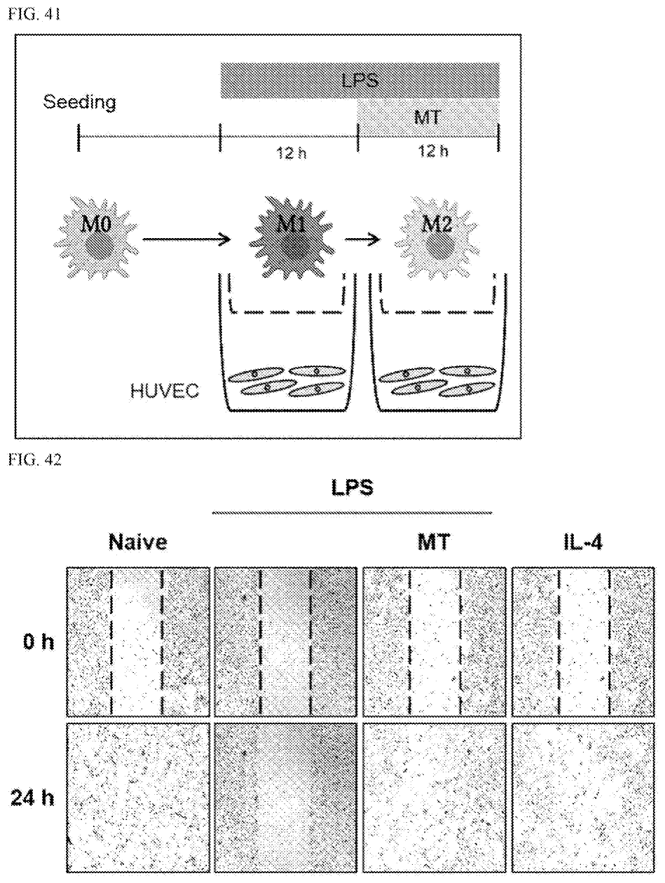

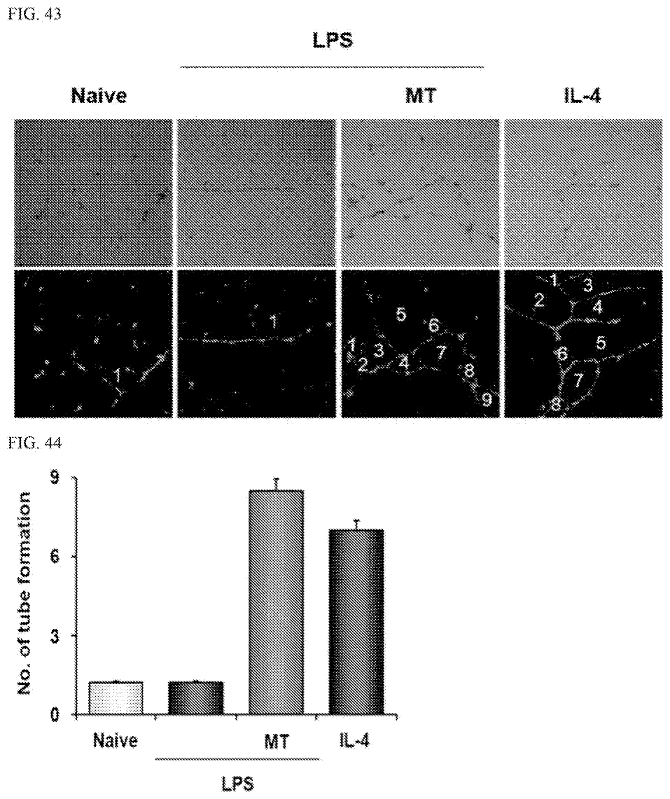

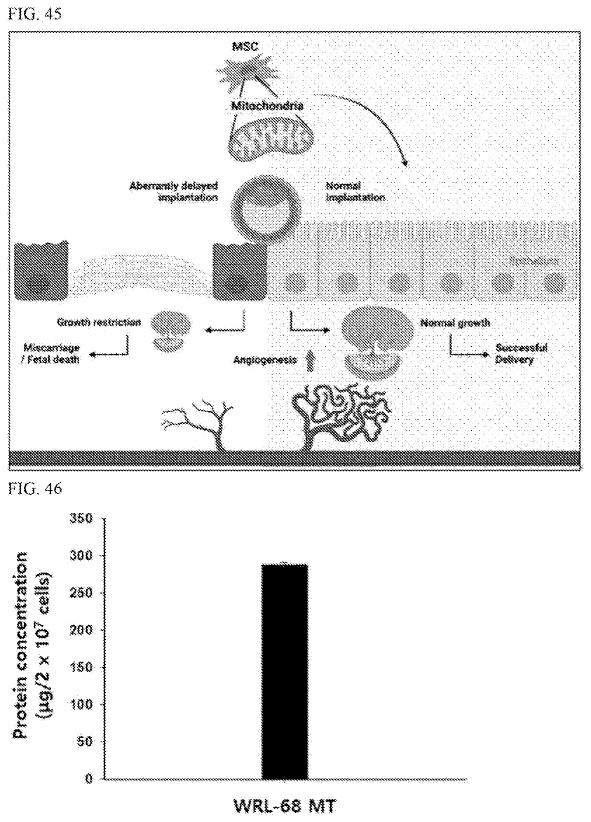

As a result, as shown in FIG. 42, it was confirmed that when co-cultured with macrophages showing M2 polarization by MT, the cell migration was promoted similarly to the control group in which M2 polarization was induced by IL-4 treatment. In addition, as shown in FIGS. 43 and 44, it was confirmed that when co-cultured with macrophages showing M2 polarization by MT, the tube formation was promoted significantly more than in the control group.

1. A pharmaceutical composition for preventing or treating Asherman's syndrome or complications thereof, comprising isolated mitochondria as an active ingredient.

2. The pharmaceutical according to claim 1, wherein the mitochondria are isolated from cells or plasma.

3. The pharmaceutical composition according to claim 2, wherein the cells are somatic cells, germ cells, stem cells, blood cells, or a combination thereof.

4. The pharmaceutical composition according to claim 3, wherein the stem cells are mesenchymal stem cells, induced pluripotent stem cells, embryonic stem cells, or a combination thereof.

5. The pharmaceutical composition according to claim 4, wherein the mesenchymal stem cells are derived from umbilical cord, umbilical cord blood, bone marrow, fat, muscle, nerve, skin, amniotic membrane, placenta, synovial fluid, testis, periosteum, or a combination thereof.

6. The pharmaceutical composition according to claim 2, wherein the plasma is plasma from bone marrow, umbilical cord blood, or peripheral blood.

7. The pharmaceutical composition according to claim 1, wherein the pharmaceutical composition reduces the expression of fibrosis factors.

8. The pharmaceutical composition according to claim 7, wherein the fibrosis factor is one or more selected from the group consisting of Col1a1, Col3a1, Timp1, and Tgfβ1.

9. The pharmaceutical composition according to claim 1, wherein the pharmaceutical composition increases the expression of one or more vascular endothelial cell markers selected from the group consisting of Hgf, Igf1, Ang1, Vegf-A, Hif1α, and Hif2α.

10. The pharmaceutical composition according to claim 1, wherein the pharmaceutical composition is a preparation for direct administration into the uterus or an injection for intravenous, intramuscular or subcutaneous administration.

11. The pharmaceutical composition according to claim 1, wherein the Asherman's syndrome complication is one or more selected from the group consisting of intrauterine adhesion, leiomyoma of uterus, endometriosis, ectopic pregnancy, miscarriage, ovarian cystic tumor, menstrual disorder, infertility, sterility, pelvic adhesion, pelvic pain, and pelvic inflammatory disorder.

12. The pharmaceutical composition according to claim 1, wherein the mitochondria in the pharmaceutical composition are included at a concentration of 0.1 μg/mL to 1,000 μg/mL.

13. A method for preventing or treating Asherman's syndrome or complications thereof, comprising administering the pharmaceutical composition according to claim 1 to a subject.

14. The method for preventing or treating Asherman's syndrome or complications thereof according to claim 13, wherein the administering step involves administering the pharmaceutical composition directly into the uterus of a subject, intravenously, intramuscularly, or subcutaneously.

15. Use of isolated mitochondria for the prevention or treatment of Asherman's syndrome or complications thereof.

16. Use of isolated mitochondria for the manufacture of a medicament for preventing or treating Asherman's syndrome or complications thereof.
